(19) United States
(12) Patent Application Publication
(10) Pub. No.: US 2026/0144474 A1
(43) Pub. Date: May 28, 2026

(54) CARDIAC INFORMATION PROCESSING SYSTEM

(71) Applicant: Acutus Medical, Inc., Carlsbad, CA (US)

(72) Inventors: Derrick Ren-yu CHOU, San Diego, CA (US); Timothy J. CORVI, Carlsbad, CA (US); Vince BURGESS, Encinitas, CA (US); Paras PARIKH, San Marcos, CA (US); Randell L. WERNETH, San Diego, CA (US); Graydon Ernest BEATTY, Carlsbad, CA (US); Nathan ANGEL, Oceanside, CA (US); Christoph SCHARF, Horgen (CH); Lam DANG, Rueschlikon (CH); R. Maxwell FLAHERTY, Topsfield, MA (US); J. Christopher FLAHERTY, Nottingham, NH (US)

(21) Appl. No.: 19/254,234

(22) Filed: Jun. 30, 2025

Related U.S. Application Data
(63) Continuation of application No. 17/048,151, now abandoned.
(60) Provisional application No. 62/668,659, filed on May 8, 2018.
(60) Provisional application No. 62/811,735, filed on Feb. 28, 2019.

(51) Int. Cl. A61B 5/367; A61B 5/00; A61B 5/0536; A61B 6/03; A61B 8/00; A61B 8/08; A61B 17/22; A61B 18/00; A61B 18/02; A61B 18/14; A61B 18/18

(52) U.S. Cl. CPC A61B 5/367; A61B 5/4848; A61B 5/7267; A61B 17/22004; A61B 18/02; A61B 18/1492; A61B 18/1815; A61B 5/0075; A61B 5/0536; A61B 6/032; A61B 6/037; A61B 8/0883; A61B 8/565; A61B 2018/0022; A61B 2018/00267; A61B 2018/00351; A61B 2018/00577

(57) ABSTRACT

Provided herein are cardiac information processing systems comprising multiple subsystems for performing a procedure and producing procedure data, and a processing module. The multiple subsystems comprise: a mapping subsystem comprising at least one mapping catheter; an imaging subsystem comprising at least one imaging device; and a treatment subsystem comprising at least one treatment device. The processing module receives the procedure data, the processing module comprising at least one processor and at least one algorithm. The at least one algorithm is configured to perform an assessment of the procedure data and produce evaluation data based on the assessment. Methods of processing cardiac information are also provided.

### CROSS REFERENCE TO RELATED APPLICATIONS

The present application is a continuation application of U.S. patent application Ser. No. 17/048,151, filed Oct. 16, 2020, Publication Number US 2021-0169394, published Jun. 6, 2021, which is a 371 national stage application of Patent Cooperation Treaty Application No. PCT/US2019/031131 filed May 7, 2019, which in turn claims priority under 35 USC 119(e) to U.S. Provisional Patent Application No. 62/668,659 filed May 8, 2018, entitled “Cardiac Information Processing System,” and to U.S. Provisional Patent Application No. 62/811,735 filed Feb. 28, 2019, entitled “Cardiac Information Processing System,” the contents of which are incorporated herein by reference.

The present application, while not claiming priority to, may be related to Patent Cooperation Treaty Application No. PCT/US2017/056064, entitled “Ablation System with Force Control”, filed Oct. 11, 2017, which claims priority to U.S. Provisional Application Ser. No. 62/406,748, entitled “Ablation System with Force Control”, filed Oct. 11, 2016, and U.S. Provisional Application Ser. No. 62/504,139, entitled “Ablation System with Force Control”, filed May 20, 2017, each of which is hereby incorporated by reference.

The present application, while not claiming priority to, may be related to Patent Cooperation Treaty Application No. PCT/US2017/030915, entitled “Cardiac Information Dynamic Display System and Method”, filed May 3, 2017, which claims priority to U.S. Provisional Patent Application Ser. No. 62/331,351, entitled “Cardiac Information Dynamic Display System and Method”, filed May 3, 2016, each of which is hereby incorporated by reference.

The present application, while not claiming priority to, may be related to U.S. application Ser. No. 14/422,941, entitled “Catheter, System and Methods of Medical Uses of Same, Including Diagnostic and Treatment Uses for the Heart”, filed Feb. 20, 2015, which is a 35 USC 371 national stage filing of Patent Cooperation Treaty Application No. PCT/US2013/057579, entitled “Catheter System and Methods of Medical Uses of Same, Including Diagnostic and Treatment Uses for the Heart”, filed Aug. 30, 2013, published as WO 2014/036439, which claims priority to U.S. Patent Provisional Application Ser. No. 61/695,535, entitled “System and Method for Diagnosing and Treating Heart Tissue”, filed Aug. 31, 2012, each of which is hereby incorporated by reference.

The present application, while not claiming priority to, may be related to U.S. application Ser. No. 14/762,944, entitled “Expandable Catheter Assembly with Flexible Printed Circuit Board (PCB) Electrical Pathways”, filed Jul. 23, 2015, which is a 35 USC 371 national stage filing of Patent Cooperation Treaty Application No. PCT/US2014/015261, entitled “Expandable Catheter Assembly with Flexible Printed Circuit Board (PCB) Electrical Pathways”, filed Feb. 7, 2014, published as WO 2014/124231, which claims priority to U.S. Patent Provisional Application Ser. No. 61/762,363, entitled “Expandable Catheter Assembly with Flexible Printed Circuit Board (PCB) Electrical Pathways”, filed Feb. 8, 2013, each of which is hereby incorporated by reference.

The present application, while not claiming priority to, may be related to U.S. patent application Ser. No. 14/865,435, entitled “Method and Device for Determining and Presenting Surface Charge and Dipole Densities on Cardiac Walls”, filed Sep. 25, 2015, which is a continuation of U.S. Pat. No. 9,167,982, entitled “Method and Device for Determining and Presenting Surface Charge and Dipole Densities on Cardiac Walls”, filed Nov. 19, 2014, which is a continuation of U.S. Pat. No. 8,918,158 (hereinafter the '158 patent), entitled “Method and Device for Determining and Presenting Surface Charge and Dipole Densities on Cardiac Walls”, issued Dec. 23, 2014, which is a continuation of U.S. Pat. No. 8,700,119 (hereinafter the '119 patent), entitled “Method and Device for Determining and Presenting Surface Charge and Dipole Densities on Cardiac Walls”, issued Apr. 15, 2014, which is a continuation of U.S. Pat. No. 8,417,313 (hereinafter the '313 patent), entitled “Method and Device for Determining and Presenting Surface Charge and Dipole Densities on Cardiac Walls”, issued Apr. 9, 2013, which was a 35 USC 371 national stage filing of PCT Application No. CH2007/000380, entitled “Method and Device for Determining and Presenting Surface Charge and Dipole Densities on Cardiac Walls”, filed Aug. 3, 2007, published as WO 2008/014629, which claimed priority to Swiss Patent Application No. 1251/06 filed Aug. 3, 2006, each of which is hereby incorporated by reference.

The present application, while not claiming priority to, may be related to U.S. patent application Ser. No. 14/886,449, entitled “Device and Method for the Geometric Determination of Electrical Dipole Densities on the Cardiac Wall”, filed Oct. 19, 2015, which is a continuation of U.S. Pat. No. 9,192,318, entitled “Device and Method for the Geometric Determination of Electrical Dipole Densities on the Cardiac Wall”, filed Jul. 19, 2013, which is a continuation of U.S. Pat. No. 8,512,255, entitled “Device and Method for the Geometric Determination of Electrical Dipole Densities on the Cardiac Wall”, issued Aug. 20, 2013, published as US2010/0298690 (hereinafter the '690 publication), which was a 35 USC 371 national stage application of Patent Cooperation Treaty Application No. PCT/IB09/00071 filed Jan. 16, 2009, entitled “A Device and Method for the Geometric Determination of Electrical Dipole Densities on the Cardiac Wall”, published as WO2009/090547, which claimed priority to Swiss Patent Application 00068/08 filed Jan. 17, 2008, each of which is hereby incorporated by reference.

The present application, while not claiming priority to, may be related to U.S. application Ser. No. 14/003,671, entitled “Device and Method for the Geometric Determination of Electrical Dipole Densities on the Cardiac Wall”, filed Sep. 6, 2013, which is a 35 USC 371 national stage filing of Patent Cooperation Treaty Application No. PCT/US2012/028593, entitled “Device and Method for the Geometric Determination of Electrical Dipole Densities on the Cardiac Wall”, published as WO2012/122517 (hereinafter the '517 publication), which claimed priority to U.S. Patent Provisional Application Ser. No. 61/451,357, each of which is hereby incorporated by reference.

The present application, while not claiming priority to, may be related to U.S. Design application Ser. No. 29/475,273, entitled “Catheter System and Methods of Medical Uses of Same, Including Diagnostic and Treatment Uses for the Heart”, filed Dec. 2, 2013, which is a 35 USC 371 national stage filing of Patent Cooperation Treaty Application No. PCT/US2013/057579, entitled “Catheter System and Methods of Medical Uses of Same, Including Diagnostic and Treatment Uses for the Heart”, filed Aug. 30, 2013, which claims priority to U.S. Patent Provisional Application Ser. No. 61/695,535, entitled “System and Method for Diagnosing and Treating Heart Tissue”, filed Aug. 31, 2012, which is hereby incorporated by reference.

The present application, while not claiming priority to, may be related to Patent Cooperation Treaty Application No. PCT/US2014/15261, entitled “Expandable Catheter Assembly with Flexible Printed Circuit Board (PCB) Electrical Pathways”, filed Feb. 7, 2014, which claims priority to U.S. Patent Provisional Application Ser. No. 61/762,363, entitled “Expandable Catheter Assembly with Flexible Printed Circuit Board (PCB) Electrical Pathways”, filed Feb. 8, 2013, which is hereby incorporated by reference.

The present application, while not claiming priority to, may be related to Patent Cooperation Treaty Application No. PCT/US2015/11312, entitled “Gas-Elimination Patient Access Device”, filed Jan. 14, 2015, which claims priority to U.S. Patent Provisional Application Ser. No. 61/928,704, entitled “Gas-Elimination Patient Access Device”, filed Jan. 17, 2014, which is hereby incorporated by reference.

The present application, while not claiming priority to, may be related to Patent Cooperation Treaty Application No. PCT/US2015/22187, entitled “Cardiac Analysis User Interface System and Method”, filed Mar. 24, 2015, which claims priority to U.S. Patent Provisional Application Ser. No. 61/970,027, entitled “Cardiac Analysis User Interface System and Method”, filed Mar. 28, 2014, which is hereby incorporated by reference.

The present application, while not claiming priority to, may be related to Patent Cooperation Treaty Application No. PCT/US2014/54942, entitled “Devices and Methods for Determination of Electrical Dipole Densities on a Cardiac Surface”, filed Sep. 10, 2014, which claims priority to U.S. Patent Provisional Application Ser. No. 61/877,617, entitled “Devices and Methods for Determination of Electrical Dipole Densities on a Cardiac Surface”, filed Sep. 13, 2013, which is hereby incorporated by reference.

The present application, while not claiming priority to, may be related to U.S. Patent Provisional Application Ser. No. 62/161,213, entitled “Localization System and Method Useful in the Acquisition and Analysis of Cardiac Information”, filed May 13, 2015, which is hereby incorporated by reference.

The present application, while not claiming priority to, may be related to U.S. Patent Provisional Application Ser. No. 62/160,501, entitled “Cardiac Virtualization Test Tank and Testing System and Method”, filed May 12, 2015, which is hereby incorporated by reference.

The present application, while not claiming priority to, may be related to U.S. Patent Provisional Application Ser. No. 62/160,529, entitled “Ultrasound Sequencing System and Method”, filed May 12, 2015, which is hereby incorporated by reference.

The present application, while not claiming priority to, may be related to U.S. Patent Provisional Application Ser. No. 62/619,897, entitled “System for Recognizing Cardiac Conduction Patterns”, filed Jan. 21, 2018, which is hereby incorporated by reference.

The present application, while not claiming priority to, may be related to U.S. Patent Provisional Application Ser. No. 62/668,647, entitled “System for Identifying Cardiac Conduction Patterns”, filed May 8, 2018, which is hereby incorporated by reference.

### FIELD OF THE INVENTION

The present invention relates generally to medical systems for processing information, and in particular systems for processing cardiac information of one or more patients.

### BACKGROUND

Cardiac signals (e.g. charge density, dipole density, voltage, etc.) vary across the endocardial surface in magnitude. The magnitude of these signals is dependent on several factors, including local tissue characteristics (e.g. healthy vs. disease/scar/fibrosis/lesion) and regional activation characteristics (e.g. “electrical mass” of activated tissue prior to activation of the local cells). A common practice is to assign a single threshold for all signals at all times across the surface. The use of a single threshold can cause low-amplitude activation to be missed or cause high-amplitude activation to dominate/saturate, leading to confusion in interpretation of the map. Failure to properly detect activation can lead to imprecise identification of regions of interest for therapy delivery or incomplete characterization of ablation efficacy (excess or lack of block).

The continuous, global mapping of atrial fibrillation yields a tremendous volume of temporally- and spatially-variable activation patterns. A limited, discrete sampling of map data may be insufficient to provide a comprehensive picture of the drivers, mechanisms, and supporting substrate for the arrhythmia. Clinician review of long durations of AF can be challenging to remember and piece together to complete the “bigger picture.”

For these and other reasons, there is a general need for systems that process cardiac information to achieve improved outcomes in patients with one or more cardiac conditions.

### SUMMARY

According to an aspect of the present inventive concepts, a cardiac information processing system, comprises multiple subsystems for performing a procedure and producing procedure data, the multiple subsystems comprising: a mapping subsystem comprising at least one mapping catheter; an imaging subsystem comprising at least one imaging device; a treatment subsystem comprising at least one treatment device; and a processing module for receiving the procedure data. The system further comprises at least one processor and at least one algorithm, and the at least one algorithm is configured to perform an assessment of the procedure data and produce evaluation data based on the assessment.

In some embodiments, the procedure data comprises cardiac procedure data.

In some embodiments, the evaluation data comprises data related to the evaluation of cardiac health and/or treatment effectiveness.

In some embodiments, the algorithm comprises a machine learning algorithm.

In some embodiments, the algorithm produces option data. The option data can comprise at least one therapeutic strategy. The therapeutic strategy can comprise an assessment of probability of success and/or an assessment of risk. The therapeutic strategy can comprise an assessment of probability of success and an assessment of risk.

In some embodiments, the system further comprises a network configured to transfer information between two or more of: the mapping system, the imaging system, the therapy system, and/or the processing unit.

In some embodiments, the system is configured to produce a functional model of the cardiac anatomy. The functional model can comprise one or more parameters selected from the group consisting of: the size and/or location of the pulmonary veins; the size, location, and/or other parameters of one or more cardiac valves; the size and/or shape of one or more cardiac chambers; the thickness of the walls of one or more cardiac chambers; the size and/or location of the atrial appendage; and combinations thereof. The functional model can comprise a triangular mesh and/or a quadratic mesh.

In some embodiments, the system is configured to predict activation wavefronts, such as atrial activation wavefronts. The system can be configured to predict the atrial activation wavefronts during regular rhythms and/or irregular rhythms. The system can be configured to predict the atrial activation wavefront during an irregular rhythm comprising atrial fibrillation. The system can be configured to record a plurality of bioelectric signals from: within one or more chambers of the heart, on the epicardial surface of the heart, and/or from the skin.

In some embodiments, the system further comprises a learning algorithm. The learning algorithm can comprise an algorithm selected from the group consisting of: an artificial intelligence algorithm; a machine learning algorithm; a deep-learning algorithm; and combinations thereof. The algorithm can be configured to analyze present activation and predict future activation pathways across a heart chamber. The algorithm can be further configured to compare the predicted activation pathways with the actual activation pathways that occur, and the system can improve the algorithm based on the comparison. The improvement can be achieved using artificial intelligence, machine learning, and/or deep-learning. The system can be configured to perform a statistical analysis that identifies preferential conduction pathways, and the learning algorithm can incorporate the identified pathways.

In some embodiments, the system further comprises a predictive algorithm configured to predict the effect of one or more treatments on a pattern of activation. The predictive algorithm can comprise an interactive search algorithm, configured to provide treatment locations and/or a minimum number of treatment steps to efficiently and/or effectively treat an abnormal rhythm. The predictive algorithm can analyze information selected from the group consisting of: bioelectric signals recorded by one or more electrodes; calculated voltage, dipole, and/or surface charge information calculated from applying an inverse solution to bioelectric signals recorded by one or more electrodes; and combinations thereof.

In some embodiments, the system further comprises an algorithm configured to perform predictive processing based on a functional model of cardiac anatomy. The functional model of cardiac anatomy can comprise a refractory time parameter. The refractory time parameter can be dependent on the cycle length interval preceding the current cycle. The refractory time parameter can comprise a frequency-dependent time parameter. The refractory time parameter can be modified by simulating the effects of one or more medications. The refractory time parameter can be configured to vary based on the region of cardiac tissue being modeled. The refractory time parameter can be configured to vary based on: the thickness of the tissue; the density of the tissue; the heterogeneity of the tissue; the percentage of fibrosis of the tissue; the number of trabeculated muscles in posterior versus anterior locations; and/or the septum of the atrium. The refractory time parameter can be adjusted based on the volume and/or the pressure within the heart chamber. The refractory time parameter can be adjusted such that the refractory time parameter can be longer during the ventricular stroke and/or shorter during systole.

In some embodiments, the system further comprises an algorithm configured to assess the amplitude and/or morphology of a recorded biopotential signal in an area of cardiac tissue. The algorithm can be configured to predict one or more tissue characteristics in the area of cardiac tissue. The algorithm can be configured to predict an area of slow conduction and/or scar tissue when signals with low amplitude are recorded. The algorithm can be configured to predict an area of healthy tissue when signals with high amplitude are recorded. The algorithm can be configured to compare signals prior to and after a tissue treatment is performed, and an effectiveness of the treatment can be predicted.

In some embodiments, the system further comprises an algorithm configured to assess morphology of data. The algorithm can be configured to perform a correlation selected from the group consisting of: a negative signal with a centrifugal activation; a positive signal with an approaching wavefront; a positive signal followed by a negative signal with a passing wavefront; a negative signal followed by a positive signal with a mirror image activation wavefront from an opposite site; opposing vectors with the collision of activation wavefronts; opposite vectors with a negative and a positive component with the incomplete block of a line; positive vectors along a line with no negative component with a complete block of a line; reverse polarity of a signal around a point in opposite directions with a focal activation at the point; loss of the negative component of an electrical signal with a transmural ablation; and combinations of these.

In some embodiments, the system is configured to produce mapping data calculated using an inverse solution method. The mapping data can comprise dipole density and/or surface charge density data. The regularization parameters of the inverse solution can be automatically adapted to provide the most stable model of activation. The mapping data can comprise dipole density data calculated from non-contact recordings and voltage measurements recorded from contact recordings, and the system can comprise an algorithm configured to compare the calculated dipole density data to the voltage measurements and can modify the parameters of the inverse solution to correct discrepancies identified between the calculated data and the measured data.

In some embodiments, the system further comprises an algorithm configured to predict the efficacy of a patient medication.

In some embodiments, the system further comprises an algorithm configured to predict the efficacy of a tissue treatment procedure. The algorithm can predict the efficacy based on a database of prior patient treatment data. The algorithm can predict the efficacy based on patient age, patient atrial diameters, and/or size of electrograms of the patient.

In some embodiments, the system further comprises an algorithm configured to predict atrial activation wavefronts based on a frequency analysis. The algorithm can be further configured to predict the atrial activation wavefronts based on a determined dominant frequency and/or cycle length. The algorithm can be further configured to predict the atrial activation wavefronts based on a parameter selected from the group consisting of: a dominant frequency; a frequency ratio; entropy; organization index; energy of the signal; power of the signal; and combinations thereof.

The technology described herein, along with the attributes and attendant advantages thereof, will best be appreciated and understood in view of the following detailed description taken in conjunction with the accompanying drawings in which representative embodiments are described by way of example.

### BRIEF DESCRIPTION OF THE DRAWINGS

FIG. 1 illustrates a schematic view of a cardiac information processing system, consistent with the present inventive concepts.

FIG. 2 illustrates a schematic view of a cardiac mapping system, consistent with the present inventive concepts.

FIG. 3 illustrates a flow chart of a method of processing cardiac information is illustrated, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the term “threshold” refers to a maximum level, a minimum level, and/or range of values correlating to a desired or undesired state. In some embodiments, a system parameter is maintained above a minimum threshold, below a maximum threshold. within a threshold range of values and/or outside a threshold range of values, to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As used herein, the term “functional element” is to be taken to include one or more elements constructed and arranged to perform a function. A functional element can comprise a sensor and/or a transducer. In some embodiments, a functional element is configured to deliver energy and/or otherwise treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise an expandable assembly. A functional assembly can comprise one or more functional elements.

As used herein, the term “processor” shall refer to a module, such as an electronic module, that receives data and performs one or more functions (e.g. mathematical functions) on the data. The term processor can refer to one or more physical processors, for example one or more electronic processing units, each comprising one or more (e.g. several) processing cores, for example one or more multi-core, multi-threaded, processors. As used herein, the term processor can refer to a processor of the system, as used for a particular purpose. This use is not intended to limit the use of the processor to a single purpose. In some embodiments, a system described herein comprises a single physical processing unit, described herein as numerous processors for various purposes. In some embodiments, processors described herein can refer to “virtual” processors, such as cloud-based processing systems, such as when the physical hardware of the system does not include a physical processor, such as when the physical hardware instructs a remote processing system (e.g. via one or more algorithms) to compute data received by the system. Processors can be configured to perform one or more calculations on data, for example, a processor can be instructed to analyze data and to parse the data into one or more subsets (e.g. parse, distribute, and/or characterize) based on one or more analyzed characteristics of the data. In some embodiments, a processor can further process (e.g. analyze) already processed data.

As used herein, the term “fluid” can refer to a liquid, gas, gel, or any flowable material, such as a material which can be propelled through a lumen and/or opening.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein. Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Provided herein are cardiac information processing systems for a patient. The systems can record cardiac and other physiologic information of a patient, as well as information related to a therapy given to a patient. The system can use the recorded information to provide information to a clinician of the patient, such as information related to one or more proposed future treatments that could be performed. In some embodiments, a future treatment includes a cardiac ablation procedure, and the system provides information related to: types of ablation to be performed; patterns of ablations to be performed; and/or locations of ablations to be performed. The systems of the present inventive concepts can include one or more subsystems, such as a mapping subsystem, imaging subsystem, and/or therapy subsystem. The mapping subsystems of the present inventive concepts can be configured to determine and/or process information related to dipole density and/or surface charge density (singly or collectively “dipole density” herein).

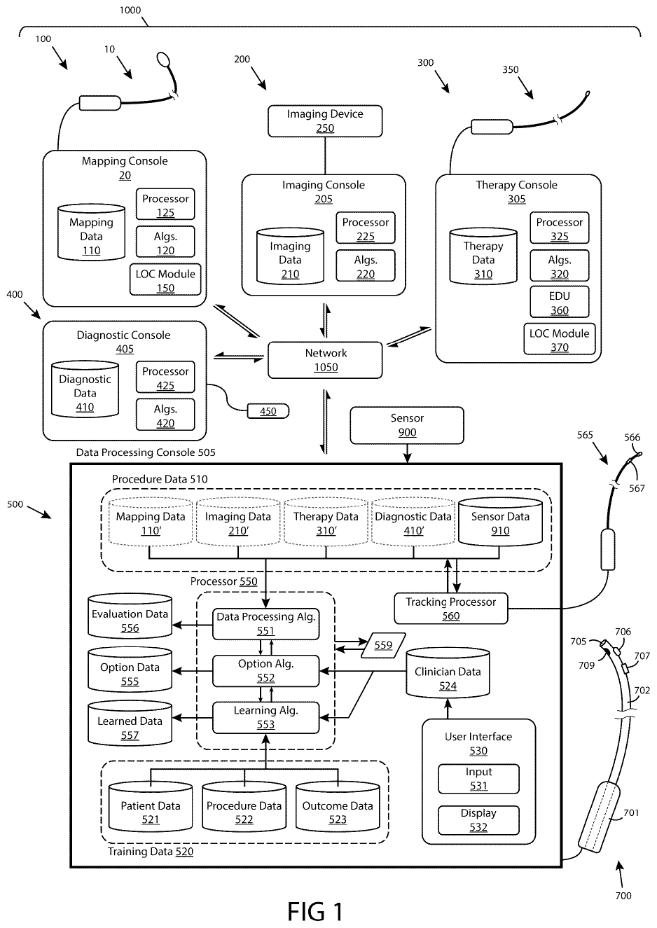

Referring now to FIG. 1, a block diagram of a cardiac information processing system is illustrated, consistent with the present inventive concepts. The cardiac information processing system, system 1000 shown, can include a mapping subsystem 100, an imaging subsystem 200, a therapy subsystem 300, and/or a data processing module 500. In some embodiments, system 1000 further comprises a diagnostic subsystem 400. One or more subsystems or other portions of system 1000 can be configured (e.g. independently configured and/or integrated with other subsystems) to perform cardiac mapping, diagnosis, prognosis, and/or treatment, such as for treating a disease or disorder of a patient, such as an arrhythmia or other cardiac condition, as described herein. In some embodiments, system 1000 analyzes recorded, calculated, processed, and/or otherwise available data (hereinafter recorded data). For example, data recorded by system 1000, such as mapping data, imaging data, therapy data, sensor data, and/or other data, as described herein, can be analyzed using one or more algorithms (e.g. one or more machine learning algorithms), to produce option data 555. Option data 555 can represent one or more procedural options (e.g. therapeutic suggestions) provided by data processing module 500 related to treating a cardiac condition of a patient, such as a therapy suggestion and/or strategy, based on recorded data as interpreted by an option algorithm, algorithm 552.

The various subsystems of system 1000 can each comprise one or more systems or devices configured to operate separately from system 1000 (e.g. operate independent of system 1000). For example, mapping subsystem 100 can comprise a mapping system configured for use with or without other components of system 1000. Similarly, imaging subsystem 200 and therapy subsystem 300 can each be configured for use independently or with other components of system 1000. Data processing module 500 can be configured to interface with one or more mapping subsystems 100, one or more imaging subsystems 200, and/or one or more therapy subsystems 300. For example, some embodiments of system 1000 include: a mapping subsystem 100 configured for non-contact dipole density mapping; an imaging subsystem 200 comprising a MRI imaging device and a fluoroscopy imaging device; and a therapy subsystem 300 configured to deliver ablation energy (e.g. ablative RF energy, cryogenic energy, ultrasound energy, and the like).

As shown in FIG. 1, data processing module 500 can comprise a console, console 505. Data processing module 500 communicates with mapping subsystem 100, imaging subsystem 200, therapy subsystem 300, and/or diagnostic subsystem 400 via a communication network, network 1050. Network 1050 can comprise a communication network selected from the group consisting of: a wireless communication network; a web-based communication network; a wired communication network, such as via one or more interconnect cables between one or more of consoles of subsystems 100, 200, 300, 400, data processing module 500; and combinations of these. Each subsystem of system 1000 can comprise a console, such as consoles 20, 205, 305, 405, and 505 described herein. In some embodiments, a single console can provide functionality for multiple of the subsystems (e.g. a single console comprises two or more of consoles 20, 205, 305, 405, and/or 505). For example, a single console (e.g. console 20 of mapping subsystem 100) can comprise the one or more components of mapping subsystem 100, as well as data processing module 500. Furthermore, a single console can include one or more of the components of mapping subsystem 100, imaging subsystem 200, therapy subsystem 300, diagnostic subsystem 400, and/or data processing module 500. A multi-function console can be configured to operably attach to one or more mapping catheters 10, imaging devices 250, therapeutic devices 350, diagnostic devices 450, and/or other devices of system 1000.

In some embodiments, system 1000 comprises one or more tissue assessment and/or characterization components (e.g. mapping subsystem 100 and/or imaging subsystem 200 configured to analyze tissue), including one or more devices and/or algorithms configured to record and process data related to the cardiac tissue of a patient. System 1000 can further comprise one or more therapy components (e.g. therapy subsystem 300), including one or more devices (e.g. treatment device 350) for delivering therapy to the cardiac tissue of the patient (e.g. ablation therapy and/or other therapy). In some embodiments, system 1000 comprises tracking processor 560, which can be configured to co-register recorded data from one or more subsystems, temporally and/or spatially, for example in both space (e.g. a volume representing a heart chamber) and time (e.g. syncing time-based recorded data received from or otherwise provided by multiple subsystems of system 1000). In some embodiments, the therapy delivery component (e.g. treatment device 350 or other therapy delivery device) delivers tissue-modifying energy (e.g. ablation energy) to a location of the body, and the one or more tissue assessment components determine (e.g. record and analyze) the characteristics of the tissue before, during, and/or after the delivery of energy. Tracking processor 560 can be configured to maintain coordinate registration between data related to therapy delivery and data related to tissue assessment, such that the tissue assessed is at a specific location, such as at the location where the therapy component delivered therapy (e.g. delivered ablative energy).

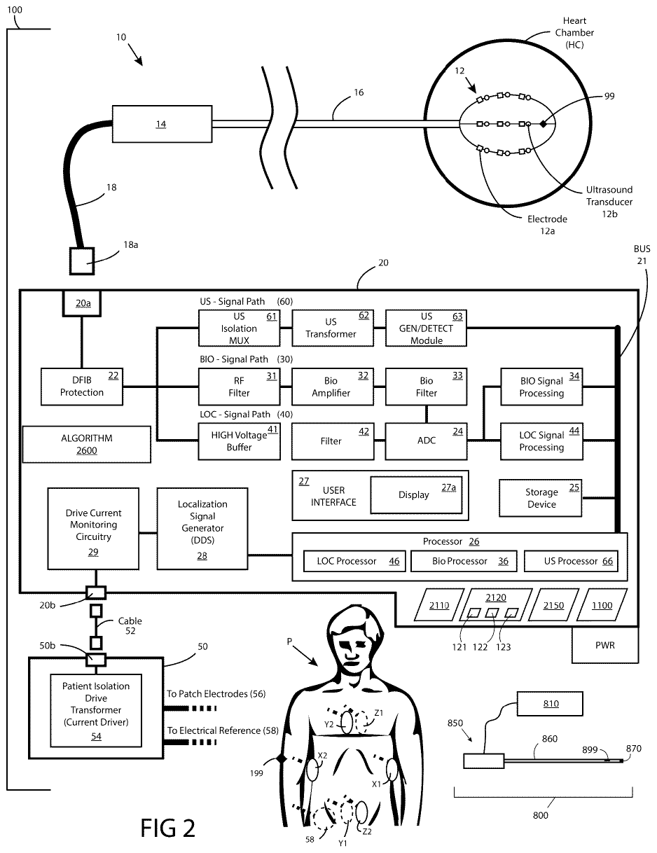

As shown further in FIG. 1, mapping subsystem 100 can comprise a console 20, and a mapping catheter 10 operably attached to console 20. Mapping subsystem 100 can record, process, analyze, and/or store cardiac data, mapping data 110. Mapping data 110 can comprise cardiac activity data (e.g. electrical and/or mechanical cardiac data) and/or cardiac anatomy data. Mapping subsystem 100 can further include one or more algorithms for processing mapping data 110, mapping algorithm 120 (e.g. for processing recorded data and determining cardiac activity), and one or more processors 125 for executing mapping algorithms 120. Mapping subsystem 100 can include a localization (LOC) module 150, configured to localize (e.g. determine the position and/or orientation of) mapping catheter 10 within the patient. LOC module 150 can be of similar construction and arrangement to localization processor 46, and associated components, as described herebelow in reference to FIG. 2. LOC module 150 is further described herebelow. Mapping subsystem 100 can be of similar construction and arrangement to mapping subsystem 100, as described herebelow in reference to FIG. 2.

Mapping subsystem 100 can comprise an electrical mapping system configured to produce mapping data 110 corresponding to: contact voltage data (e.g. voltage recorded by an electrode in contact with the cardiac wall); non-contact voltage data (e.g. voltage data calculated from voltage recordings made at locations offset from the cardiac wall); non-contact dipole density data (e.g. dipole density data calculated from voltage recordings made at locations offset from the cardiac wall); hybrid voltage data (e.g. voltage data recorded and/or calculated using contact and non-contact measurements); hybrid dipole density data (e.g. dipole density data calculated using contact and non-contact measurements); body surface measurement data; activation timing data (e.g. unipolar and/or bipolar activation timing data calculated from recorded electrocardiograms); and combinations of these. Additionally or alternatively, mapping subsystem 100 can be configured to determine and/or analyze cardiac structure and/or function as well as cardiac activity data, for example, structural and/or functional characteristics selected from the group consisting of: wall motion; wall thickness; ejection fraction; chamber volume; and combinations of these.

Imaging subsystem 200 comprises a console 205, and an imaging device 250 operably attached to console 205. Imaging subsystem 200 can record, process, analyze, and/or store image data, imaging data 210. Imaging subsystem 200 can further include one or more algorithms for processing imaging data 210, imaging algorithm 220 (e.g. an algorithm for processing recorded image data and producing a 3D model of the cardiac anatomy), and one or more processors 225 for executing algorithms 220. Imaging device 250 can comprise a device selected from the group consisting of: an optical imaging device, such as a spectroscopy, interferometry, OCT, or near infrared optical imaging device; an ultrasonic imaging device, such as a 2D or 3D, ultrasonic imaging device; an impedance-based imaging device such as an impedance tomography device; an MRI imaging device, such as a Late Gadolinium Enhancement, T1, T2, or DTI MRI imaging device; a CT imaging device; a PET imaging device; a SPECT imaging device; a fluoroscope or other X-Ray imaging device; intracardiac echo (ICE); transesophageal echo (TEE); transthoracic echo (TTE); and combinations of these. In some embodiments imaging device 250 can comprise all or a portion of another device of system 1000, and/or can be operably attached to another device of system 1000, for example an optical fiber-based imaging device, attached to and/or integrated into mapping catheter 10. Imaging device 250 can utilize an imaging modality selected from the group consisting of: ultrasound; visible light; infrared light; near-infrared light; magnetic resonance; transmission-based imaging modalities (such as X-ray or CT); reflective-based imaging modalities; emission-based imaging modalities (such as PET or SPECT); and combinations of these. Imaging device 250 can comprise an imaging mode selected from the group consisting of: a narrow field of view mode, such as to image a localized region, such as to image a small portion of tissue; a wide field of view mode, such as to image a body cavity, one or more chambers of the heart, and/or a portion of the heart; a functional imaging mode, such as to capture fluid flow, velocity, displacement and/or volume; and combinations of these. Imaging device 250 can be configured to image from a location selected from the group consisting of: outside the body; within a body cavity or structure, but outside of a heart chamber; within a heart chamber; and combinations of these. In some embodiments, at least a portion of imaging device 250 can be in a fixed position relative to a heart chamber, and/or it can be free to move relative to a heart chamber, for example manually moved within a chamber of the heart by the user. In some embodiments, imaging algorithm 220 is configured to analyze imaging data 210 to determine the characteristics of the imaged tissue (e.g. to diagnose fibrosis and/or other tissue abnormalities). In some embodiments algorithm 220 can distinguish between viable and necrotic tissue. Additionally or alternatively, algorithm 220 can be configured to analyze image data recorded over time (e.g. video data) to characterize heart motion, ejection fraction, and/or other tissue movement-based cardiac characteristics. Cardiac characterization information can be stored as imaging data 210.

In some embodiments, imaging subsystem 200 comprises an ultrasound system. The ultrasound system can include an array of transducers positioned external to the body. The array can be a contiguous grid array (1D, 1.5D, or 2D) configured to perform conventional phased-array imaging. In some embodiments, the physical dimensions of the array can be configured to image through the space between a pair of ribs, such as to avoid shadowing. Additionally or alternatively, the array can be distributed into two or more sub-arrays, or individual elements, for example, to image between different sets of ribs or from multiple positions, or fully-distributed around the torso, such as when configured as a wearable garment. The ultrasound system can be configured to operate in a pulse-echo mode and/or in a ‘pitch-catch’ mode, where the transmitting elements, sub-array, or array are located in a location different than the receiving elements, sub-array, or array for a given transmission event. The ultrasound system can be configured to operate with parallel receive processing, multiple transmit beams (simultaneous or in quick, patterned succession), or gating, to optimize timing. The ultrasound system can be adapted for use within the body, either external to the heart chamber (e.g. transthoracic or transesophageal echocardiography, TTE or TEE, respectively) or internal to the heart chamber or circulatory system (e.g. intracardiac echocardiography ICE or intravascular ultrasound IVUS).

Therapy subsystem 300 comprises a console 305, and a therapeutic device 350 operably attached to console 305. Therapy subsystem 300 can include data recorded and/or generated (e.g. calculated) by therapy subsystem 300, therapy data 310. Therapy subsystem 300 can further include one or more algorithms for processing therapy data 310, therapy algorithm 320 (e.g. for determining energy level and/or duration of therapy to be delivered), and one or more processors 325 for executing therapy algorithm 320. Therapy subsystem 300 can include a localization (LOC) module 370, configured to localize (e.g. determine the position and/or orientation of) therapy device 350 within the patient. LOC module 370 can be of similar construction and arrangement to localization processor 46, and associated components, as described herebelow in reference to FIG. 2. LOC module 370 is further described herebelow. Therapy subsystem 300 can further include an energy delivery unit, EDU 360. In some embodiments, therapy subsystem 300 comprises a treatment modality selected from the group consisting of: RF ablation; cryogenic ablation; light energy ablation, such as laser ablation; pulsed field ablation, such as electroporation ablation; acoustic ablation, such as high intensity focused ultrasound or low intensity collimated ultrasound ablation; microwave ablation; surgical intervention; robotically controlled and/or assisted therapy; radiation therapy; mechanical stabilization; and combinations of these. Therapy subsystem 300 can be of similar construction and arrangement to treatment device 850 and energy delivery unit 810, as described herebelow in reference to FIG. 2. Therapeutic device 350 can be configured to treat tissue by modifying one or more tissue characteristics (e.g. modifying the conductive characteristics of the tissue).

In some embodiments, therapy subsystem 300 comprises a radio frequency (RF) ablation system 300a, including an RF ablation device 350a. RF device 350a can be an irrigated or non-irrigated device. RF therapy subsystem 300a can be configured to deliver mono-polar, bipolar, and/or multi-polar RF energy. RF device 350a can comprise one or more electrodes for delivering ablative energy to the tissue of the patient (e.g. energy delivered from an RF energy-providing EDU 360a). RF device 350a can include a device selected from the group consisting of: a single tip electrode device; a device with an array of electrodes, such as a basket or balloon deployed array of electrodes; and combinations of these. RF therapy subsystem 300a can be configured to provide a closed loop delivery of RF energy (e.g. via a power, temperature, impedance, contact, and/or force controlled loop).

In some embodiments, therapy subsystem 300 comprises a cryogenic ablation system 300b, including a cryogenic ablation device 350b. Cryogenic device 350b can include a device selected from the group consisting of: a cryogenic balloon; a point source cryogenic delivery element; a linear cryogenic delivery element; a shape configurable cryogenic delivery element; and combinations of these. EDU 360 can comprise a cryogenic energy delivery unit, EDU 360b, including a gas exchange unit, a near super critical unit, and/or a super critical unit.

In some embodiments, system 1000 comprises diagnostic system 400. Diagnostic subsystem 400 can comprise a console 405, and a diagnostic device 450 operably attached to console 405. Diagnostic subsystem 400 can store data recorded and/or generated (e.g. calculated) by diagnostic subsystem 400, diagnostic data 410. Diagnostic subsystem 400 can further include one or more algorithms for processing diagnostic data 410, diagnostic algorithm 420, and one or more processors 425 for executing diagnostic algorithm 420. In some embodiments, diagnostic subsystem 400 can include one or more localization components, as described herein. Diagnostic system 400 can be configured to provide diagnostic data related to one or more conditions of the patient, such as one or more cardiac conditions of the patient. Diagnostic device 450 can be configured to be implanted and/or otherwise inserted in the patient, and/or remain external to the patient. In some embodiments, diagnostic system 400 is configured to record a patient parameter selected from the group consisting of: blood pressure; blood glucose; a blood gas parameter; a blood flow parameter; respiration; pH; and combinations of these. In some embodiments, diagnostic system 400 is configured to record patient genetic information.

Sensor 900 can comprise one or more sensors selected from the group consisting of: an electrode or other sensor for recording electrical activity; a force sensor; a pressure sensor; a magnetic sensor; a motion sensor; a velocity sensor; an accelerometer; a strain gauge; a physiologic sensor; a glucose sensor; a pH sensor; a blood sensor; a blood gas sensor; a blood pressure sensor; a flow sensor; an optical sensor; a spectrometer; an interferometer; a measuring sensor, such as to measure size, distance, and/or thickness; a tissue assessment sensor; and combinations of these. Sensor 900 can comprise one or more sensors positioned on one or more components of system 1000, such as when mapping catheter 10, therapeutic device 350, and/or another catheter device of system 1000 comprises a sensor 900 for positioning one or more sensors within the patient (e.g. within the heart of the patient). Alternatively or additionally, sensor 900 can comprise one or more sensors positioned on the skin of the patient and/or otherwise external but proximate the patient. Sensor 900 can comprise one or more sensors that record information related to cardiac function, such as cardiac motion.

Data processing module 500 can comprise a specialized computer, including one or more data processing modules, a power module, a user input module, a user output module, and one or more storage and/or memory modules, each of which are operably connected to form a unified system. Data processing module 500 can comprise one or more data repositories (e.g. one or more databases of data stored in memory, such as volatile or non-volatile memory). Module 500 can record, process, analyze, and/or store data recorded intraoperatively, procedure data 510. Procedure data 510 can comprise various data related to one or more procedures performed on a patient using one or more subsystems or other components of system 1000. For example, procedure data 510 can comprise mapping data 110′ (e.g. mapping data 110 of subsystem 100), imaging data 210′ (e.g. imaging data 210 of subsystem 200), therapy data 310′ (e.g. therapy data 310 of subsystem 300), and/or diagnostic data 410′ (e.g. diagnostic data 410 of subsystem 400) (each referred to collectively herein as data 110, 210, 310, and 410, respectively). In some embodiments, subsystem data (e.g. mapping data 110 from mapping subsystem 100 and/or other subsystem provided data) is copied to data processing module 500 over network 1050, and accessed by the one or more processors of module 500 via local storage (e.g. mapping data 110′). Additionally or alternatively, subsystem data (e.g. mapping data 110), is stored in a single location (e.g. mapping data 110 or mapping data 110′), and is streamed or otherwise accessed by various processors of system 1000 via network 1050. In some embodiments, any subsystem or other component of system 1000 (e.g. mapping subsystem 100, imaging subsystem 200, therapy subsystem 300, diagnostic subsystem 400, and/or module 500) can access data stored on any other subsystem or component (e.g. via network 1050).

Procedure data 510 can comprise data recorded during a single clinical procedure, and/or over multiple procedures performed on a single patient. The multiple procedures can comprise multiple cardiac treatment and/or diagnostic procedures. For example, the multiple procedures can comprise an imaging procedure (e.g. an MRI) and a cardiac treatment procedure. Procedure data 510 can further include data recorded by sensor 900 of system 1000. Data processing module 500 can be configured to analyze a first set of data (e.g. procedure data 510), and produce a second set of data based on the first set. For example, as describe in further detail herebelow, data processing module 500 can analyze procedure data 510, and produce evaluation data 556 and/or option data 555.

Module 500 can include a co-registration processor, tracking processor 560. Tracking processor 560 can be configured to maintain and/or calculate proper registration of data recorded temporally and/or spatially from two or more subsystems of system 1000. In some embodiments, imaging subsystem 200 produces imaging data 210 comprising 2D and/or 3D image data. Imaging data 210 can comprise static and/or time varying (e.g. video) data, which can be provided in 2D and/or 3D imaging modalities. Mapping subsystem 100 can produce mapping data 110 comprising recorded cardiac activity data and/or device position data (e.g. data representing the 3D position of mapping catheter 10) stored with respect to a location in 3D space and/or time (e.g. data representing where and/or when, respectively, the cardiac activity data was recorded). In some embodiments, mapping subsystem 100 comprises an imaging capability, such that mapping data 110 further comprises image data, similar to or dissimilar from imaging data 210. Therapy subsystem 300 can produce therapy data 310 comprising therapy delivery data, which can be stored with respect to a location in 3D space and/or a time (e.g. where and/or when, respectively, therapy was delivered to a patient). Tracking processor 560 can be configured to correlate the temporal and/or spatial components of mapping data 110, imaging data 210, and/or therapy data 310, such that data processing module 500 can process data within a single spatial coordinate system and/or it can process data that spans a single timeline.

In some embodiments, LOC module 150 and LOC module 370 of mapping subsystem 100 and therapy subsystem 300, respectively, comprise similar localization modalities. In some embodiments, LOC module 150 and LOC module 370 comprise dissimilar localization modalities. LOC modules 150, 370 can each comprise modalities selected from the group consisting of: impedance-based localization; magnetic-based localization; any modality for localizing components in or around a body or body chamber; and combinations of these. LOC modules 150, 370 can each utilize transmitted, emitted and/or reflected energy modalities, such as ultrasound, RF, and/or fluoroscopy. In some embodiments, LOC module 150 or LOC module 370 (or any other localization module of system 1000) is chosen as a primary LOC module. In these embodiments, tracking processor 560 can transpose localization data recorded by any or all secondary localization modules to the coordinate and/or temporal data to that of the data recorded by the primary module. Alternatively or additionally, tracking processor 560 can establish a unique, independent coordinate system, and transpose all localization data to be co-registered within the unique, independent coordinate system.

In some embodiments, each LOC module (e.g. modules 150, 370) of system 1000 uses the same method of localization (e.g. impedance-based localization). In these embodiments, data recorded by a first LOC module (e.g. module 150), can comprise information related to the location of a device other than the primary device of the first subsystem (e.g. mapping subsystem 100). For example, LOC module 150 can localize therapy device 350, in addition to mapping catheter 10, if both subsystems 100 and 300 employ impedance-based localization methods. Alternatively or additionally, a first LOC module of system 1000 can enable more than one method of localization (e.g. impedance-based and magnetic-based localization methods), and the first module can localize devices using both modalities. Tracking processor 560 can use this data that is common between the localization of different subsystems to co-register the data, and/or to improve the accuracy of the co-registration performed by other means (e.g. a “best fit” registration based on anatomic geometry). In some embodiments, system 1000 further comprises a co-registration device, tracking catheter 565, comprising two or more localization elements, a first localization element 566 and a second localization element 567 as shown. First and second localization elements 566, 567 can comprise dissimilar elements configured to be localized by different modalities. For example, first localization element 566 can comprise an electrode configured to be localized using an impedance-based system, and second localization element 567 can comprise a coil, such as an electromagnetic coil, and/or a magnet, which can be configured to be localized using a magnetic-based localization system. Tracking catheter 565 can comprise a known geometry (e.g. size and/or shape of at least the distal portion of tracking catheter 565), such that tracking processor 560 can use the localization data of tracking catheter 565 gathered by two dissimilar localization modules to co-register the data. For example, if a primary or first localization module employs an impedance-based localization and a second localization module employs a magnetic-based localization, if tracking catheter 565 is capable of being localized by either impedance or magnetic systems, it can be used by tracking processor 560 to unify the coordinate systems of both localization modules. Alternatively or additionally, a device of system 1000 (e.g. mapping catheter 10) can comprise a “multi-modal” device, for example a device including electrodes and ultrasound transducers that can be localized using impedance and ultrasonic methods. This multi-modal device can be used to correlate two independent localization modalities of system 1000. In some embodiments, an ultrasonically enabled internal device (single or multi-modal) can receive ultrasound energy from external to the body, enabling localization of the external transmitter.

System 1000 can comprise a patient introduction device, sheath 700, for example a transseptal introducer sheath. Sheath 700 can be configured to slidingly receive one or more catheters and/or other elongate devices (e.g. sequentially or simultaneously) for introduction into a heart chamber. Sheath 700 can comprise a handle 701, positioned on a proximal portion of a shaft 702, extending distally from handle 701. Shaft 702 can comprise one or more lumens extending therethrough, each lumen configured to slidingly receive one or more elongate devices. Sheath 700 can be configured to introduce mapping catheter 10, therapeutic device 350, catheter-based diagnostic device 450, and/or a catheter-based imaging device 250. Sheath 700 can comprise one or more functional elements, such as functional elements 706 and 707 shown. Functional elements 706, 707 can comprise similar or dissimilar localization elements, as described hereabove, configured to localize sheath 700 within a patient. In some embodiments, functional elements 706 and/or 707 comprise one or more electrode-based localization elements, such as to perform impedance-based localization. In some embodiments, functional elements 706 and/or 707 comprise one or more coils or magnets (“coils” herein), such as to perform magnetic-based localization. In some embodiments, functional elements 706 and/or 707 comprise at least one electrode and at least one coil (e.g. a magnet or an electromagnetic coil). In some embodiments, a device of system 1000 (e.g. a device not comprising localization elements) are introduced into a patient through sheath 700, and subsequently localized via one or more functional elements 706, 707. In some embodiments, one or more functional elements 706 and/or 707 comprise a sensor and/or transducer, as described herein.

In some embodiments, sheath 700 comprises a fixation element, lock 709. Lock 709 can be configured to be deployed, or otherwise activated, to frictionally or otherwise engage a device inserted through lumen 705, such as to fix the position of the device relative to sheath 700. Lock 709 can comprise an expandable balloon, configured to restrict lumen 705, capturing the inserted device (e.g. a balloon attached to an inflation lumen and port, not shown). Lock 709 can comprise a functional mechanism selected from the group consisting of: a magnetic lock; an expandable lock; a friction increasing mechanism; a deployable hook; and combinations of these.

User interface 530 comprises a user interface including one or more user input and/or user input components. User interface 530 can comprise one or more user input components such as one or more components selected from the group consisting of: button, knob, lever; foot switch; eye gaze tracker; microphone; keyboard; touchscreen; and combinations of these. User interface 530 can comprise one or more user output components such as one or more components selected from the group consisting of: a visual transducer such as a display, a touchscreen display and/or a light; an audio transducer such as a speaker; a tactile transducer such as a vibrational transducer; and combinations of these. Generally, user interface can comprise at least user input 531, and display 532.

In some embodiments, one or more subsystems (e.g. 100, 200, 300, and/or 400) of system 1000 comprises one or more user interface components, such as one or more user input devices and/or one or more user output devices, such as one or more displays. Additionally or alternatively, module 500 can be configured to incorporate the input and/or output functions of any or all of system 1000 subsystems into user interface 530 (e.g. when user interface 530 is configured as a “master” user interface). In some embodiments user interface 530 is configured to display (e.g. on display 532) graphical or other representations of mapping data 110, imaging data 210, therapy data 310, evaluation data 556 and/or option data 555. Module 500 can be configured to display data as: a single value; a trace or a waveform displayed with respect to time; an image; an image with varying parameters, such as parameters represented by varying color and/or brightness; an image overlaid onto another image or rendering; and combinations of these. The various display configurations provided by system 1000 can be of similar construction and arrangement as described in applicant's co-pending applications: International Patent Application Serial Number PCT/US2017/030915, titled “CARDIAC INFORMATION DYNAMIC DISPLAY SYSTEM AND METHOD” filed, May 3, 2017; International Patent Application Serial Number PCT/US2017/030922, titled “CARDIAC MAPPING SYSTEM WITH EFFICIENCY ALGORITHM” filed May 3, 2017; and U.S. Patent Provisional Application Ser. No. 62/668,647, titled “SYSTEM FOR IDENTIFYING CARDIAC CONDUCTION PATTERNS”, filed May 8, 2018; the contents of each of which are incorporated herein by reference in their entirety for all purposes.

As a non-limiting, illustrative example, therapy device 350 can be localized using an impedance or magnetic-based method, and imaging device 250 can be an ultrasound imaging device. Display 532 can be configured to graphically render intracardiac devices (e.g. device 350) that have been localized and also render a representation of the heart chamber surface (e.g. in a 3D coordinate system in one or more views). An ultrasound planar or volumetric image of the cardiac tissue at the location of therapy delivery (e.g. at the tip of device 350) can be overlaid onto the graphical rendering. Tracking processor 560 can co-register the recorded data for the display. Tracking processor 560 can utilize the spatial location of the ultrasound reflection from therapy device 350 detected by imaging device 250 simultaneously with the impedance-based location measured directly by LOC module 370 to co-register the coordinate systems of the two subsystems. In the example, the displayed ultrasound image can be limited to a size representative of the expected regional effect of the therapy to be delivered. For an RF ablation lesion, for example, the desired affected area can be approximately 5 mm in diameter. An ultrasound volume of approximately 5 mm in any direction can be displayed on the rendered chamber surface proximate the tip of therapy device 350. Prior to the delivery of therapy (e.g. before ablation energy is delivered), the ultrasound image can show the tissue density and/or reflectance (as an example) of the untreated chamber wall as an indexed brightness level, color, or other graphical indicia. The average density of the imaged tissue prior to therapy can be calculated and stored as a baseline or calibration reference for subsequent measurements (e.g. stored as image data 210). As therapy is delivered, the resultant change in the tissue density and/or reflectance can also be shown with indexed graphical indicia. After therapy has been delivered (e.g. after ablation energy has been delivered to tissue), the tissue density and/or reflectance can continue to be assessed for changes occurring in the tissue after therapy (e.g. for a period of time after therapy). A previously untreated region of tissue, (e.g. a region of tissue near the site of therapy delivery) can be automatically or manually selected by system 1000 or a user, respectively, and an overlay of ultrasound imaging can be displayed for this tissue region (e.g. displayed simultaneously with the ultrasound at the site of therapy delivery). The tissue of the untreated region can be assessed simultaneously and the tissue density and/or reflectance can be displayed with its own indexed graphical indicia. A calculation of the average density of the tissue at the untreated region can be used as a common-mode measurement to improve the accuracy and/or sensitivity of measurements made at the site of therapy.

The preceding is a non-limiting example of a system of devices utilized for recording and displaying recorded information relating to the efficacy of a treatment of tissue. Any combination described herein of various mapping subsystems, imaging subsystems, and therapy subsystems can be used to evaluate, image, treat or otherwise record or alter the properties of tissue, and the resultant data can be displayed to the user.

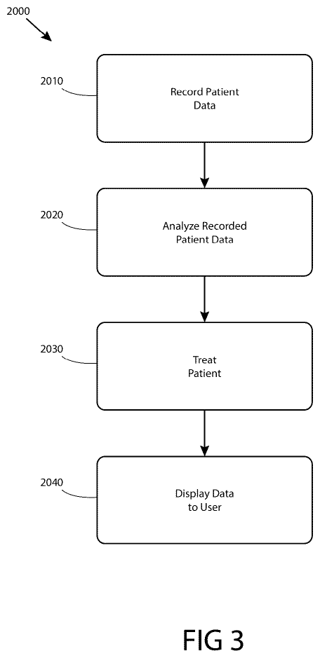

Data processing module 500 can comprise a processor, processor 550. Module 500 can include a data processing algorithm 551, configured to analyze procedure data 510 using processor 550 and produce evaluation data 556. In some embodiments, evaluation data 556 includes a classification of one or more portions of cardiac tissue (e.g. a classification of the health of the tissue). For example, data processing algorithm 551 can analyze one or more of mapping data 110, imaging data 210, and/or sensor data 910, and classify portions of tissue as healthy tissue, fibrotic tissue, scar tissue, and/or other indicators of the condition of the particular portion of tissue. In some embodiments, evaluation data 556 includes a classification of disease or phenotype identified in a portion of tissue. Evaluation data 556 can include classifying a tissue characteristic selected from the group consisting of: density; stiffness; toughness; electrical impedance; acoustic impedance; radiographic and/or metabolic absorption; metabolic performance; and combinations of these. Tissue characteristics can be determined at a point, in a line or path, in a plane or surface, and/or in a volume. Tissue characteristics can be associated with a specificity of no more than 10 mm, or no more than 5 mm, 2.5 mm, or 1 mm. Data processing algorithm 551 can identify portions of tissue that have been previously treated (e.g. ablated, such as by analyzing the recorded data such as therapy data 310), such as to identify one or more tissue portions to be analyzed. Data processing algorithm 551 can be configured to assess the effectiveness of the treatment (e.g. at the one or more tissue portions identified as having been treated). In some embodiments, data processing algorithm 551 can be configured to assess a user-selected region of tissue. In some embodiments, data processing algorithm 551 is configured as described herebelow in reference to FIG. 3.

In some embodiments, evaluation data 556 includes cardiac functionality data. For example, data 556 can include data corresponding to the electrical functionality, mechanical functionality, ejection fraction, and/or hemodynamics of the heart or heart chamber. In some embodiments, data processing algorithm 551 is similar to one or more algorithms described in applicant's co-pending U.S. Patent Provisional Application Ser. No. 62/668,647, titled “SYSTEM FOR IDENTIFYING CARDIAC CONDUCTION PATTERNS”, filed May 8, 2018, the content of which is incorporated herein by reference in its entirety for all purposes. In some embodiments, evaluation data 556 includes the results of quantitative or qualitative processing of procedure data 510, including: calculated dimensions; rate of change per unit time; spatial volume; percentage of a physiologic or other parameter, such as the percentage of the chamber wall thickness; difference between two quantities; quantitative relationship to a threshold; and combinations of these. In some embodiments, evaluation data 556 can comprise complexity data, as described herebelow in reference to FIG. 2. One or more processors of data processing unit 500 can perform a function selected from the group consisting of: data aggregation; co-registration of data, such as spatial or temporal alignment of data; command and/or control of one or more subsystems of system 1000 to facilitate the interaction or interfacing between the subsystems, each of which may include independent subsystems; and combinations of these.

In some embodiments, system 1000 is configured to produce a functional model of the cardiac anatomy, functional model 559. Functional model 559 can comprise a digital model of the cardiac anatomy, based off of data recorded by system 1000, such as mapping data 110, imaging data 210, diagnostic data 410, sensor data 910, and/or data calculated by system 1000, such as evaluation data 556, option data 555, and learned data 557. Functional model 559 can be analyzed, such as by an algorithm of system 1000, such as to predict treatment efficacy. Functional model 559 can be configured as described herebelow in reference to FIG. 3.

In some embodiments, data processing algorithm 551 is configured to determine a quantitative value and/or assess a qualitative state of a parameter of a portion of tissue. Assessed tissue parameters can include parameters selected from the group consisting of: electrical; mechanical; physiological; electrophysiological; histological; functional; dynamic; thermodynamic; chemical; biochemical; and combinations of these. Data processing algorithm 551 can be configured to subdivide procedure data 510 into multiple sub-regions (e.g. multiple portions of tissue represented by data 510). Data processing algorithm 551 can be configured to perform mathematical processing on one or more than one of these sub-regions, such as one or more sub-regions within a larger region (e.g. a user selected region of tissue). Mathematical processing can include a statistical function across one or more sub-regions, such as: a mean, median, minimum or maximum, percentile; a time varying function across one or more sub-regions, such as a derivative or an integral with respect to time; a spatially varying function across one or more sub-regions, such as a spatial variance or distance weighted function; and combinations of these. Algorithm 551 can be configured to assign a “state” to a sub-region of tissue, for example a true or false value determined by a measurement, a measurement against one or more thresholds, and/or a measurement compared to a separate measurement in time or space (e.g. from a different location and/or time). In some embodiments, a sub-region can be volume of tissue defined as a surface area of tissue, with an unknown or assumed depth (e.g. cardiac wall thickness), and/or a sub-region can include a specific depth (e.g. such that one “area” of tissue can be subdivided into two or more separate volumes within the cardiac wall). In some embodiments, the size and/or shape of the sub-regions can be defined by tissue characteristics. For example, a first sub-region can be defined by algorithm 551 as a region of ablated tissue, and a second sub-region is defined a region of untreated tissue.

Module 500 can be configured to accept clinician input (such as information entered via input 531), which is stored as clinician data 524. Clinician data 524 can comprise clinician preferences, such as preferences used to bias one or more algorithms of module 500. For example, as described herebelow, option algorithm 552 can provide a set of therapeutic options to the clinician based on procedure data 510. The options provided (e.g. option data 555) can be biased, for example, towards a preferred therapeutic technique as indicated by clinician data 524. In some embodiments, clinician data can comprise data corresponding to historical information regarding the clinician, for example therapeutic success rates, therapeutic trends, and/or other data gathered over multiple procedures, for one or more patients. This data can be manually entered by the clinician, and/or stored and recalled by system 1000 or another data storage and distribution system, such as a digital patient history file repository maintained by a clinical center. Clinician data can inform option algorithm 552 and learning algorithm 553 as described further herebelow.

Module 500 can include option algorithm 552, configured to analyze evaluation data 556, as well as procedure data 510 and clinician data 524, and produce option data 555. Option data 555 can include therapeutic strategy data (e.g. suggestions to the clinician on one or more proposed clinical approaches for treatment based on the analyzed data). Option data 555 can also include probabilistic, as well as predictive, outcomes of the therapeutic strategies proposed. This data can include the probability of post therapy function, including arrhythmia burden, rhythm conversion, and/or recurrence. Module 500 can be configured to display (e.g. on display 532) one or more therapeutic strategies, along with calculated “pros and cons” of each strategy. In some embodiments, the patient's clinician implements a proposed therapeutic strategy, and/or the clinician proceeds with another treatment option, and therapy data 310 is updated with the actual procedures performed. System 1000 can be configured to continuously or intermittently update procedure data 510 (e.g. during and/or post treatment), such that evaluation data 556 and/or option data 555 can be updated and therapeutic strategies can be adjusted (e.g. and provided to the clinician) in a closed loop fashion.

In some embodiments, option algorithm 552 comprises a recursive algorithm configured to model the cardiac tissue (e.g. from evaluation data 556), and simulate one or more treatment options, subsequently simulating remodeled conduction patterns based on the simulated treatment, and analyzing the simulated results. In a recursive manner, one or more treatment options are then simulated and the process is repeated. Algorithm 552 can be configured to perform several iterations, such as several thousands of iterations, such as hundreds of thousands (i.e. more than 100,000 iterations), of simulated treatment options, analysis, and results. These iterations can be used to generate one, two, three or more treatment strategy options, including one or more treatment steps. The treatment strategy options can be selected (via algorithm 552) from the set of simulated therapeutic options analyzed. The selection can be based on a calculated probability of success of the strategy. The calculated probability can be displayed to the clinician along with the proposed therapeutics steps.

Additionally or alternatively, option algorithm 552 can perform a risk assessment for a suggested treatment strategy (for example by calculating any risk associated with the proposed treatment steps). The risk assessment can also be displayed to the clinician. In some embodiments, a risk/reward ratio (e.g. a ratio defined by the potential risk as compared to the likelihood of success) can be displayed for each treatment strategy option. In some embodiments, clinician data 524 can comprise clinician preferences related to therapeutic strategies, for example preferences selected from the group consisting of: anatomic regions to avoid treating, such as the posterior wall; anatomic regions to tend away from treating; treatment patterns to tend towards, such lines or “X” patterns; treatment patterns to avoid; treatment durations and/or energy to avoid; prioritization of therapeutic result, such as achieving sinus rhythm with the fewest treatment steps vs maintaining sinus rhythm regardless of treatment steps; a weight or other prioritization of one or more variables considered by option algorithm 552; and combinations of these. In some embodiments, option algorithm 552 is configured as described herebelow in reference to FIG. 3.

In some embodiments, module 500 comprises an artificial intelligence (AI) system and/or a machine learning system, including a learning algorithm 553. Module 500 can comprise historic data, for example data recorded over multiple procedures, for multiple patients, including patient data 521, procedure data 522, and/or outcome data 523. Collectively this historic data can comprise a set of data provided to learning algorithm 553, training data 520. Patient data 521 can include parameters of one, two, or more patients previously treated for a cardiac disease, including parameters selected from the group consisting of: current age; age at the time of a procedure; disease state; height; weight; health and wellness information; hereditary information; genetic information; medication history; dietary history; activity history; and combinations of these. Procedure data 522 can include information related to one or more procedures performed on one or more patients (e.g. the patients represented in patient data 521), including information selected from the group consisting of: cardiac activity data recorded during a procedure; cardiac function data recorded during a procedure; type of treatment delivered during a procedure (e.g. treatment modality); amount of treatment delivered during a procedure; location of treatment delivered during a procedure, such as where tissue was ablated during a procedure; attending clinician; hospital or clinic where procedure occurred; and combinations of these. Outcome data 523 can include information related to the outcome of the procedures represented by procedure data 522, including information selected from the group consisting of: overall procedural success; cardiac rhythm post procedure; patient comfort during and/or post procedure; patient follow up data, such as data collected at a patient follow up; efficacy of procedural outcome; and combinations of these. Training data 520 can include data collected by one or more systems 1000, for example multiple systems in use at multiple locations over a period of time. For example, in some embodiments system 1000 comprises multiple systems 1000. The amount of training data 520 can grow over time as data is collected, and distributed to modules 500 deployed in various clinics or hospitals. System 1000 data can be distributed via the internet, or by other remote data transfer protocols, and/or can be updated physically, such as by a technician.

Learning algorithm 553 can be configured to analyze training data 520, using one or more machine learning or other artificial intelligence methods, to determine trends, patterns, correlations, and/or other statistical variations within training data 520, to produce learned data 557. Learned data 557 can comprise one or more correlations between: disease state, treatment modality, and procedural success; disease state and attending clinician; treatment modality and attending clinician; treatment (e.g. ablation) pattern and procedural success; any statistically relevant correlation between patient data 521, procedure data 522, and outcome data 523; and combinations of these. Option algorithm 552 can be configured to use learned data 557 in the calculation of option data 555, as described hereabove. Learning algorithm 553 can continuously (e.g. intraoperatively) update learned data 557 by analyzing one or more of procedure data 510, evaluation data 556, clinician data 524, and or option data 555, along with training data 520. In some embodiments, clinician data 524 can alter learned data 557, for example if a clinician instructs learning algorithm 553 to only evaluate a subset of training data 520, such as only data generated during procedures the current clinician presided over. In some embodiments, learning algorithm 553 is configured as described herebelow in reference to FIG. 3.

Referring now to FIG. 2, a block diagram of an embodiment of a cardiac information processing system is illustrated, consistent with the present inventive concepts. The cardiac information processing system, mapping subsystem 100 shown, can be or include a system configured to perform cardiac mapping, diagnosis, prognosis, and/or treatment, such as for treating a disease or disorder of a patient, such as an arrhythmia or other cardiac condition as described herein. Additionally or alternatively, mapping subsystem 100 can be a system configured for teaching and or validating devices and methods of diagnosing and/or treating cardiac abnormalities or disease of a patient P. Mapping subsystem 100 can further be used for generating displays of cardiac activity, such as dynamic displays of active wave fronts propagating across surfaces of the heart. In some embodiments, mapping subsystem 100 produces diagnostic results 1100. Diagnostic results 1100 represent diagnostic data related to a cardiac condition of a patient, such as diagnostic results based on a complexity assessment as described herein.

Mapping subsystem 100 includes a mapping catheter 10, a cardiac information console 20, and a patient interface module 50 that can be configured to cooperate (e.g. collectively cooperate) to accomplish the various functions of the mapping subsystem 100. Mapping subsystem 100 can include a single power supply (PWR), which can be shared by console 20 and the patient interface module 50. Use of a single power supply in this way can greatly reduce the chance for leakage currents to propagate into the patient interface module 50 and cause errors in localization (e.g. the process of determining the location of one or more electrodes within the body of patient P). Console 20 includes bus 21 which electrically and/or otherwise operatively connects various components of console 20 to each other, as shown in FIG. 2.

Mapping catheter 10 includes an electrode array 12 that can be percutaneously delivered to a heart chamber (HC). In this embodiment, the array of electrodes 12 has a known spatial configuration in three-dimensional (3D) space. For example, in an expanded state the physical relationship of the electrode array 12 can be known or reliably assumed. Electrode array 12 can include at least one electrode 12a, or at least three electrodes 12a. Diagnostic mapping catheter 10 also includes a handle 14, and an elongate flexible shaft 16 extending from handle 14. Attached to a distal end of shaft 16 is the electrode array 12, such as a radially expandable and/or compactable assembly. In this embodiment, the electrode array 12 is shown as a basket array, but the electrode array 12 could take other forms in other embodiments. In some embodiments, expandable electrode array 12 can be constructed and arranged as described in reference to applicant's International PCT Patent Application Serial Number PCT/US2013/057579, titled “SYSTEM AND METHOD FOR DIAGNOSING AND TREATING HEART TISSUE,” filed Aug. 30, 2013, and International PCT Patent Application Serial Number PCT/US2014/015261, titled “EXPANDABLE CATHETER ASSEMBLY WITH FLEXIBLE PRINTED CIRCUIT BOARD,” filed Feb. 7, 2014, the content of each of which is incorporated herein by reference in its entirety for all purposes. In other embodiments, expandable electrode array 12 can comprise a balloon, radially deployable arms, spiral array, and/or other expandable and compactible structure (e.g. a resiliently biased structure).

Shaft 16 and expandable electrode array 12 are constructed and arranged to be inserted into a body (e.g. an animal body or a human body, such as the body of Patient P), and advanced through a body vessel, such as a femoral vein and/or other blood vessel. Shaft 16 and electrode array 12 can be constructed and arranged to be inserted through an introducer (not shown, but such as a transseptal sheath), such as when electrode array 12 is in a compacted state, and slidingly advanced through a lumen of the introducer into a body space, such as a chamber of the heart (HC), such as the right atrium or the left atrium, as examples.

Expandable electrode array 12 can comprise multiple splines (e.g. multiple splines resiliently biased in the basket shape shown in FIG. 2), each spline having a plurality of electrodes 12a and/or a plurality of ultrasound (US) transducers 12b. Three splines are visible in FIG. 2, but the basket array is not limited to three splines, more or less splines can be included in the basket array. Each electrode 12a can be configured to record (e.g. record, measure, and/or sense, herein) a bio-potential (also referred to as “electrical activity” herein), such as the voltage level at a location on a surface of the heart and/or at a location within a heart chamber HC. Recorded electrical activity is stored by mapping subsystem 100 as electrical activity data 2120a. Mapping subsystem 100 can perform one or more calculations on the recorded data 2120a to produce calculated electrical activity data 2120b. Electrical activity data 2120 can comprise recorded electrical activity data 2120a and/or calculated electrical activity data 2120b. Calculated electrical activity data 2120b can comprise data selected from the group consisting of: voltage data; mathematically processed voltage data (e.g. data that is averaged, integrated, sorted, had minimum and/or maximum values determined, and/or otherwise is mathematically processed); surface charge data; dipole density data; timing data of electrical events; filtered electrical data; electrical pattern and/or template data; an image formed by electrical values at multiple locations; and combinations of one, two, or more of these. As used herein, the term dipole density, surface charge, and surface charge density, shall be used interchangeably.

Calculated electrical activity data 2120b can comprise data that represents instances of electrical activation (also referred to as “activation” herein) of heart tissue, activation timing data 121. In some embodiments, calculated electrical activity data 2120b comprises data that represents, conduction velocity, conduction velocity data 122, and/or conduction divergence, conduction divergence data 123. Calculated electrical activity data 2120b can be correlated to one or more locations of the heart, referred to as a vertex (single location) and vertices (multiple locations) herein. In some embodiments, calculated electrical activity data 2120b comprises data selected from the group consisting of: electrical differences (e.g. deltas); averages; weighted averages; patterns and/or templates; degree-of-fit (e.g. best-fit) to one or more patterns or templates; “flow” between two or more images formed by electrical values at multiple locations (e.g. as calculated by an optical flow algorithm, such as Horn-Schunck algorithm); data analytics and/or statistics techniques, such as classification or categorization, of electrical activity using a training data set (e.g. separately acquired data, such as historical data); a computationally-optimized fit (e.g. machine learning or predictive analysis, such as by neural network or deep learning, cluster analysis); and combinations of one, two, or more of these. The calculated electrical activity data 2120b can comprise a probabilistic model that uses one or more of the aforementioned methods as inputs.

In some embodiments, activation is determined by an algorithm (e.g. an activation detection algorithm) which can include: comparing electrical data to a threshold; measurement of the slope and/or maximum and/or minimum of the electrical data; comparing electrical data at one location to electrical data at one or more nearby locations (e.g. weighted comparison); and combinations of these. In some embodiments, the activation detection algorithm can be of similar construction and arrangement as described in reference to applicant's International PCT Patent Application Serial Number PCT/US2017/030915, titled “CARDIAC INFORMATION DYNAMIC DISPLAY SYSTEM AND METHOD”, filed May 3, 2017, and International PCT Patent Application Serial Number PCT/US2017/030922, titled “CARDIAC MAPPING SYSTEM WITH EFFICIENCY ALGORITHM”, filed May 3, 2017, the content of each of which is incorporated herein by reference in its entirety for all purposes. To promote the spatial continuity for a propagation history map, the activation detection algorithm can comprise two parallel lines considering both raw signal (e.g. dipole density data and/or voltage data) together with a spatial Laplacian signal. In some embodiments, the activation detection algorithm further includes conduction velocity as one consideration of selecting between potential activation timings, as well as developing voting schemes on multiple features, such as gradient, spatial Laplacian, peak amplitude, and/or other such features.

Expanding upon the conduction velocity addition to the activation detection, the problem can be represented as a cost function with either regularization on the conduction velocity or as an inequality constraint on the conduction velocity. In some embodiments, the activation detection algorithm creates a gaussian probability distribution function around each detected activation where the highest probability is at the currently detected activation. Given no constraints, maximizing the probability of activation for every channel can output a propagation history. Alternatively, including at least one constraint can limit the solution to comprise a physiologically reasonable conduction (e.g., less than 2 m/s) and can be configured to shift the activations slightly from the currently selected activation times. Below shows an example of how the cost function can be written with constrained conduction velocity:

$$max ( {\sum }_{i = 1}^{\# of Vert} P ( i , {τ}_{i} ) ) , s . t . Conduction {Velocity}_{i} < Constant \qquad ( 1 )$$

where P is the probability of activation occurring at a particular vertex, i, at time, τ. The conduction velocity calculation is dependent on τ.

In some embodiments, the activation detection algorithm comprises a local minimum of temporal derivative of unipolar electrograms with a minimum separation between activations set to a time threshold (e.g. between 50-150 ms)

In some embodiments, the activation detection algorithm comprises a local minimum or maximum of bipolar or Laplacian electrograms with a minimum separation between activations set to a time threshold (e.g. between 50-150 ms)

In some embodiments, the activation detection algorithm comprises standard filtering with a bandpass of (0.5 to 1 Hz)-(100-300 Hz), or after an aggressive band pass of (10-30 Hz)-(100-300 Hz).

In some embodiments, the activation detection algorithm comprises a local minimum and/or maximum of temporal derivative of bipolar electrograms or Laplacian electrograms with a minimum separation between activations set to a time threshold (e.g. between 50-150 ms). The activation detection algorithm can further comprise standard filtering with a bandpass of (0.5 to 1 Hz)-(100-300 Hz) and/or an aggressive band pass of (10-30 Hz)-(100-300 Hz).

In some embodiments, the activation detection algorithm comprises zero crossings of Laplacian electrograms after a negative deflection with a minimum separation between activations set to a time threshold (e.g. between 50-150 ms)

In some embodiments, the activation detection algorithm comprises local maximums of Hilbert transformed electrograms (Phase Mapping) with a minimum separation between activations set to a time threshold (e.g. between 50-150 ms)

Each US transducer 12b can be configured to transmit an ultrasound signal and receive ultrasound reflections to determine the range to a reflecting target such as at a point on the surface of a heart chamber (HC), to provide anatomic data used in a digital model creation of the anatomy. Recorded ultrasound data and/or other anatomic data can be stored by mapping subsystem 100 as anatomic data 2110. Electrical activity data 2120 (e.g. including activation timing data 121, conduction velocity data 122, and/or conduction divergence data 123) and/or anatomic data 2110 can be stored in memory of mapping subsystem 100, for example storage device 25 described herebelow.

As a non-limiting example, three electrodes 12a and three US transducers 12b are shown on each spline in this embodiment. However, in other embodiments, the basket array can include more or less electrodes and/or more or less US transducers. Furthermore, the electrodes 12a and transducers 12b can be arranged in pairs. Here, one electrode 12a is paired with one transducer 12b, with multiple electrode-transducer pairs per spline. The inventive concept is not, however, limited to this particular electrode-transducer arrangement. In other embodiments, not all electrodes 12a and transducers 12b need to be arranged in pairs, some could be arranged in pairs while others are not arranged in pairs. Also, in some embodiments, not all splines comprise the same arrangement of electrodes 12a and transducers 12b. Additionally, in some embodiments, electrodes 12a are arranged on a first set of splines, while transducers 12b are arranged on a second set of splines. Array 12 can comprise at least four electrodes 12a, such as at least 24 electrodes 12a, such as at least 48 electrodes. Array 12 can comprise at least three splines, such as at least four splines, such as at least six splines.

In some embodiments, a second catheter, mapping catheter 10′, is used in conjunction with mapping catheter 10, for example a basket or other array of electrodes of mapping catheter 10′ can be positioned in a separate heart chamber to simultaneously map more than one chamber of the heart. Mapping catheter 10′ can be of similar or dissimilar construction to mapping catheter 10, described herein. The electrode array of mapping catheter 10′ can be arranged in a different configuration than the electrode array 12 of mapping catheter 10. For example, the array of mapping catheter 10′ can only have 24 electrodes and no US transducers while array 12 of mapping catheter 10 possesses 48 electrodes and 48 US transducers. Mapping catheter 10 and/or 10′ can comprise two or more electrode arrays, such as array 12 shown, and a second array, positioned proximal to array 12 (e.g. on shaft 16 of mapping catheter 10 or 10′).

Mapping catheter 10 can comprise a cable or other conduit, such as cable 18, configured to electrically, optically, and/or electro-optically connect mapping catheter 10 to console 20 via connectors 18a and 20a, respectively. In some embodiments, cable 18 comprises a mechanism selected from the group consisting of: a cable such as a steering cable; a mechanical linkage; a hydraulic tube; a pneumatic tube; and combinations of one or more of these.

Patient interface module 50 can be configured to electrically isolate one or more components of console 20 from patient P (e.g. to prevent undesired delivery of a shock or other undesired electrical energy to patient P). The patient interface module 50 can be integral with console 20 and/or it can comprise a separate discrete component (e.g. separate housing), as is shown. Console 20 comprises one or more connectors 20b, each comprising a jack, plug, terminal, port, or other custom or standard electrical, optical, and/or mechanical connector. In some embodiments, the connectors 20b are terminated to maintain desirable input impedance over RF frequencies, such as 10 kilohertz to 20 megahertz. In some embodiments, the termination is achieved by terminating the cable shield with a filter. In some embodiments, the terminating filters provide high input impedance in one frequency range, for example to minimize leakage at localization frequencies, and low input impedance in a different frequency range, for example to achieve maximum signal integrity at ultrasound frequencies. Similarly, the patient interface module 50 includes one or more connectors 50b. At least one cable 52 connects the patient interface module 50 with console 20, via connectors 20b and 50b.

In this embodiment, the patient interface module 50 includes an isolated localization drive system 54, a set of patch electrodes 56, and one or more reference electrodes 58. The isolated localization drive system 54 isolates localization signals from the rest of mapping subsystem 100 to prevent current leakage (e.g. signal loss) resulting in performance degradation. In some embodiments, the isolation of the localization signals from the remainder of the system comprises a range of impedance greater than 100 kiloohms, such as approximately 500 kiloohms at the localization frequencies. The isolation of the localization drive system 54 can minimize drift in localization positions and maintain a high degree of isolation between axes (as described herebelow). The localization drive system 54 can operate as a current, voltage, magnetic, acoustic, or other type of energy modality drive. The set of patch electrodes 56 and/or one or more reference electrodes 58 can consist of conductive electrodes, coils (e.g. magnets and/or electromagnetic coils), acoustic transducers, and/or other type of transducer or sensor based on the energy modality employed by the localization drive system 54. Additionally, the isolated localization drive system 54 maintains simultaneous output on all axes (e.g. a localization signal is present on each axis electrode pair, while also increasing the effective sampling rate at each electrode position). In some embodiments, the localization sampling rate comprises a rate between 10 kHz and 20 MHz, such as a sampling rate of approximately 625 kHz.

In some embodiments, the set of patch electrodes 56 include three (3) pairs of patch electrodes: an “X” pair having two patch electrodes placed on opposite sides of the ribs (X1, X2); a “Y” pair having one patch electrode placed on the lower back (Y1) and one patch electrode placed on the upper chest (Y2); and a “Z” pair having one patch electrode placed on the upper back (Z1) and one patch electrode placed on the lower abdomen (Z2). The patch electrode 56 pairs can be placed on any orthogonal and/or non-orthogonal sets of axes. In the embodiment of FIG. 2, the placement of electrodes is shown on patient P, where electrodes on the back are shown in dashed lines.

The reference patch electrode 58 can be placed on the lower back/buttocks. Additionally, or alternatively, a reference catheter 58′ (not shown but such as a percutaneous catheter including one or more electrodes and/or coils) can be placed within a body vessel, such as a blood vessel in and/or proximate the lower back/buttocks.

The placement of electrodes 56 defines a coordinate system made up of three axes, one axis per pair of patch electrodes 56. In some embodiments, the axes are non-orthogonal to a natural axis of the body, i.e., non-orthogonal to head-to-toe, chest-to-back, and side-to-side (e.g. rib-to-rib). The electrodes can be placed such that the axes intersect at an origin, such as an origin located in the heart. For instance, the origin of the three intersecting axes can be centered in an atrial volume. Mapping subsystem 100 can be configured to provide an “electrical zero” that is positioned outside of the heart, such as by locating a reference electrode 58 such that the resultant electrical zero is outside of the heart (e.g. to avoid crossing from a positive voltage to a negative voltage at one or more locations being localized).

As described above, a patch pair can operate differentially, such as when neither patch electrode 56 in a pair operates as a reference electrode, and are both driven by mapping subsystem 100 to generate the electrical field between the two. Alternatively or additionally, one or more of the patch electrodes 56 can serve as the reference electrode 58, such that they operate in a single ended mode. One of any pair of patch electrodes 56 can serve as the reference electrode 58 for that patch pair, forming a single-ended patch pair. One or more patch pairs can be configured to be independently single-ended. One or more of the patch pairs can share a patch as a single-ended reference or can have the reference patches of more than one patch pair electrically connected.

Through processing performed by console 20, the axes can be transformed (e.g. rotated) from a first orientation (e.g. a non-physiological orientation based on the placement of electrodes 56) to a second orientation. The second orientation can comprise a standard Left-Posterior-Superior (LPS) anatomical orientation, such as when the “x” axis is oriented from right to left of the patient, the “y” axis is oriented from the anterior to posterior of the patient, and the “z” axis is oriented from caudal to cranial of the patient. Placement of patch electrodes 56 and the non-standard axes defined thereby can be selected to provide improved spatial resolution when compared to patch electrode placement resulting in a normal physiological orientation of the resulting axes (e.g. due to preferred tissue characteristics between electrodes 56 in the non-standard orientation). For example, non-standard electrode 56 placement can result in reducing the negative effects of the low-impedance volume of the lungs on the localization field. Furthermore, electrode 56 placement can be selected to create axes which pass through the body of the patient along paths of equivalent, or at least similar, lengths. Axes of similar length will possess more similar energy density per unit distance within the body, yielding a more uniform spatial resolution along such axes. Transforming the non-standard axes into a standard orientation can provide a more straightforward display environment for the user. Once the desired rotation is achieved, each axis can be scaled, such as when made longer or shorter, as needed. The rotation and scaling are performed based on comparing pre-determined (e.g. expected or known) electrode array 12 shape and relative dimensions, with measured values that correspond to the shape and relative dimensions of the electrode array in the patch electrode established coordinate system. For example, rotation and scaling can be performed to transform a relatively inaccurate (e.g. uncalibrated) representation into a more accurate representation. Shaping and scaling the representation of the electrode array 12 can adjust, align, and/or otherwise improve the orientation and relative sizes of the axes for far more accurate localization.

The electrical reference electrode(s) 58 can be or at least include a patch electrode and/or an electrical reference catheter 58′, which can function as a patient “analog ground” reference. A patch electrode 58 can be placed on the skin, and can act as a return for current for defibrillation (e.g. provide a secondary purpose). An electrical reference catheter 58′ can include a unipolar reference electrode used to enhance common mode rejection. The unipolar reference electrode, or other electrodes on a reference catheter 58′, can be used to measure, track, correct, and/or calibrate physiological, mechanical, electrical, and/or computational artifacts in a cardiac signal. In some embodiments, these artifacts are due to respiration, cardiac motion, and/or artifacts induced by applied signal processing, such as filters. Another form of an electrical reference catheter 58′ can be an internal analog reference electrode, which can act as a low noise “analog ground” for all internal catheter electrodes. Each of these types of reference electrodes can be placed in relatively similar locations, such as near the lower back in an internal blood vessel (as a catheter) and/or on the lower back (as a patch). In some embodiments, mapping subsystem 100 comprises a reference catheter 58′ including a fixation mechanism (e.g. a user activated fixation mechanism), which can be constructed and arranged to reduce displacement (e.g. accidental or otherwise unintended movement) of one or more electrodes of the reference catheter 58′. The fixation mechanism can comprise a mechanism selected from the group consisting of: spiral expander; spherical expander; circumferential expander; axially actuated expander; rotationally actuated expander; and combinations of two or more of these.

In some embodiments, console 20 includes a defibrillation (DFIB) protection module 22 connected to connector 20a, which is configured to receive cardiac information from the mapping catheter 10. The DFIB protection module 22 is configured to have a precise clamping voltage and a reduced (e.g. minimum) capacitance. Functionally, the DFIB protection module 22 acts a surge protector, configured to protect the circuitry of console 20 during application of high energy to the patient, such as during defibrillation of the patient (e.g. using a standard defibrillation device).

The DFIB protection module 22 can be coupled to three signal paths, a bio-potential (BIO) signal path 30, a localization (LOC) signal path 40, and an ultrasound (US) signal path 60. Generally, the BIO signal path 30 filters noise and preserves the recorded bio-potential data, and also enables the bio-potential signals to be read (e.g. successfully recorded) while ablating (e.g. delivery of RF energy to tissue). Generally, the LOC signal path 40 allows high voltage inputs, while filtering noise from received localization data. Generally, the US signal path 60 acquires range data from the physical structure of the anatomy using the ultrasound transducers 12b for generation of a 2D or 3D digital model of the heart chamber HC, which can be stored in memory.

The BIO signal path 30 includes an RF filter 31 coupled to the DFIB protection module 22. In this embodiment, the RF filter 31 operates as a low-pass filter having a high input impedance. The high input impedance is preferred in this embodiment because it minimizes the loss of voltage from the source (e.g. mapping catheter 10), thereby better preserving the received signals (e.g. during RF ablation). The RF filter 31 is configured to allow bio-potential signals from the electrodes 12a on mapping catheter 10 to pass through RF filter 31 (e.g. passing frequencies less than 500 Hz), such as frequencies in the range of 0.5 Hz to 500 Hz. However, high frequencies, such as high voltage signals used in RF ablation, are filtered out from the bio-potential signal path 30. RF filter 31 can comprise a corner frequency between 10 kHz and 50 kHz.

A BIO amplifier 32 can comprise a low noise single-ended input amplifier that amplifies the RF filtered signal. A BIO filter 33 (e.g. a low pass filter) filters noise out of the amplified signal. BIO filter 33 can comprise an approximately 3 kHz filter. In some embodiments, BIO filter 33 comprises an approximately 7.5 kHz filter, such as when mapping subsystem 100 is configured to accommodate pacing of the heart (e.g. to avoid significant signal loss and/or degradation during pacing of the heart).

BIO filter 33 can include differential amplifier stages used to remove common mode power line signals from the bio-potential data. This differential amplifier can implement a baseline restore function which removes DC offsets and/or low frequency artifacts from the bio-potential signals. In some embodiments, this baseline restore function comprises a programmable filter which can comprise one or more filter stages. In some embodiments, the filter includes a state dependent filter. Characteristics of the state dependent filter can be based on a threshold and/or other level of a parameter (e.g. voltage), with the filter rate varied based on the filter state. Components of the baseline restore function can incorporate noise reduction techniques such as dithering and/or pulse width modulation of the baseline restore voltage. The baseline restore function can also determine, by measurement, feedback, and/or characterization, the filter response of one or more stages. The baseline restore function can also determine and/or discriminate the portions of the signal representing a physiological signal morphology from an artifact of the filter response and computationally restore the original morphology, or a portion thereof. In some embodiments, the restoration of the original morphology can include subtraction of the filter response directly and/or after additional signal processing of the filter response, such as via static, temporally-dependent, and/or spatially-dependent weighting, multiplication, filtering, inversion, and combinations of these. In some embodiments, the baseline restore function is implemented in BIO filter 33, BIO processor 36, or both.

The LOC signal path 40 includes a high voltage buffer 41 coupled to the DFIB protection module 22. In this embodiment, the high voltage buffer 41 is configured to accommodate the relatively high voltages used in treatment techniques, such as RF ablation voltages. For example, the high voltage buffer can have ±100V power-supply rails. In some embodiments, each high voltage buffer 41 has a high input impedance, such as an impedance of 100 kiloohms to 10 megaohms at the localization frequencies. In some embodiments, all high voltage buffers 41, taken together as a total parallel electrical equivalent, also has a high input impedance, such as an impedance of 100 kiloohms to 10 megaohms at the localization frequencies. In some embodiments, the high voltage buffer 41 has a bandwidth that maintains good performance over a range of high frequencies, such as frequencies between 100 kilohertz and 10 megahertz, such as frequencies of approximately 2 megahertz. In some embodiments, the high voltage buffer 41 does not include a passive RF filter input stage, such as when the high voltage buffer 41 has a ±100V power-supply. A high frequency bandpass filter 42 can be coupled to the high voltage buffer 41, and can have a passband frequency range of about 20 kHz to 80 kHz for use in localization. In some embodiments, the filter 42 has low noise with unity gain (e.g. a gain of 1 or about 1).

The US signal path 60 comprises an US isolation multiplexer, MUX 61, a US transformer with a Tx/Rx switch, US transformer 62, a US generation and detection module 63, and an US signal processor 66. The US isolation MUX 61 is connected to the DFIB protection module 22, and is used for turning on/off the US transducers 12b, such as in a predetermined order or pattern. The US isolation MUX 61 can be a set of high input impedance switches that, when open, isolate the US system and remaining US signal path elements, decoupling the impedance to ground (through the transducers and the US signal path 60) from the input of the LOC and BIO paths. The US isolation MUX 61 also multiplexes one transmit/receive circuit to one or more multiple transducers 12b on the mapping catheter 10. The US transformer 62 operates in both directions between the US isolation MUX 61 and the US generation and detection module 63. US transformer 62 isolates the patient from the current generated by the US transmit and receive circuitry in module 63 during ultrasound transmission and receiving by the US transducers 12b. The US transformer 62 can be configured to selectively engage the transmit and/or receive electronics of module 63 based on the mode of operation of the transducers 12b, for example by using a transmit/receive switch. That is, in a transmit mode, the module 63 receives a control signal from a US processor 66 (within a data processor 26) that activates the US signal generation and connects an output of the Tx amplifier to US transformer 62. The US transformer 62 couples the signal to the US isolation MUX 61 which selectively activates the US transducers 12b. In a receive mode, the US isolation MUX 61 receives reflection signals from one or more of the transducers 12b, which are passed to the US transformer 62. The US transformer 62 couples signals into the receive electronics of the US generation and detection module 63, which in-turn transfers reflection data signals to the US processor 66 for processing and use by the user interface 27 and display 27a. In some embodiments, processor 66 commands MUX 61 and US transformer 62 to enable transmission and reception of ultrasound to activate one or more of the associated transducers 12b, such as in a predetermined order or pattern. The US processor 66 can include, as examples, detection of a single, first reflection, the detection and identification of multiple reflections from multiple targets, the determination of velocity information from Doppler methods and/or from subsequent pulses, the determination of tissue density information from the amplitude, frequency, and/or phase characteristics of the reflected signal, and combinations of one or more of these.

An analog-to-digital converter (ADC) 24 is coupled to the BIO filter 33 of the BIO signal path 30 and to the high frequency filter 42 of the LOC signal path 40. Received by the ADC 24 is a set of individual time-varying analog bio-potential voltage signals, one for each electrode 12a. These bio-potential signals have been differentially referenced to a unipolar electrode for enhanced common mode rejection, filtered, and gain-calibrated on an individual channel-by-channel basis, via BIO signal path 30. Received by the ADC 24 is also a set of individual time-varying analog localization voltage signals for each axis of each patch electrode 56, via LOC signal path 40, which are output to the ADC 24 as a collection of 48 (in this embodiment) localization voltages measured at a single time for the electrodes 12a. The ADC 24 has high oversampling to allow noise shaping and filtering, e.g. with an oversampling rate of about 625 kHz. In some embodiments, sampling is performed at or above the Nyquist frequency of mapping subsystem 100. The ADC 24 is a multi-channel circuit that can combine BIO and LOC signals or keep them separate. In one embodiment, as a multi-channel circuit, the ADC 24 can be configured to accommodate 48 localization electrodes 12a and 32 auxiliary electrodes (e.g. for ablation or other processes), for a total of 80 channels. In other embodiments, more or less channels can be provided. In FIG. 2, for example, almost all of the elements of console 20 can be duplicated for each channel (e.g. except for the UI system 27). For example, console 20 can include a separate ADC for each channel, or an 80 channel ADC. In this embodiment, signal information from the BIO signal path 30 and the LOC signal path 40 are input to and output from the various channels of the ADC 24. Outputs from the channels of the ADC 24 are coupled to either the BIO signal processing module 34 or the LOC signal processing module 44, which pre-process their respective signals for subsequent processing as described herein. In each case, the preprocessing prepares the received signals for the processing by their respective dedicated processors discussed herebelow. The BIO signal processing module 34 and the LOC signal processing module 44 can be implemented in firmware, in whole or in part, in some embodiments.

The bio-potential signal processing module 34 can provide gain and offset adjustment and/or digital RF filtering having a non-dispersive low pass filter and an intermediate frequency band. The intermediate frequency band can eliminate ablation and localization signals. The bio-potential signal processing module 34 can also include digital bio-potential filtering, which can optimize the output sample rate.

Additionally, the bio-potential signal processing module 34 can also include “pace blanking”, which is the blanking of received information during a timeframe when, for example, a physician is “pacing” the heart. Temporary cardiac pacing can be implemented via the insertion or application of intracardiac, intraesophageal, and/or transcutaneous pacing leads, as examples. The goal in temporary cardiac pacing can be to interactively test and/or improve cardiac rhythm and/or hemodynamics. To accomplish the foregoing, active and passive pacing trigger and input algorithmic trigger determinations can be performed (such as by mapping subsystem 100). The algorithmic trigger determination can use subsets of channels, edge detection and/or pulse width detection to determine if pacing of the patient has occurred. Optionally, pace blanking can be applied by mapping subsystem 100 on all channels or subsets of channels, including channels on which detection did not occur.

Additionally, the bio-potential signal processing module 34 can also include specialized filters that remove ultrasound signals and/or other unwanted signals (e.g. artifacts from the bio-potential data). In some embodiments, to perform this filtering, edge detection, threshold detection and/or timing correlations are used.

The localization signal processing module 44 can provide individual channel/frequency gain calibration, IQ demodulation with tuned demodulation phase, synchronous and continuous demodulation (without MUXing), narrow band R filtering, and/or time filtering (e.g. interleaving, blanking, etc.), as discussed herebelow. The localization signal processing module 44 can also include digital localization filtering, which optimizes the output sample rate and/or frequency response.

In this embodiment, the algorithmic computations for the BIO signal path 30, LOC signal path 40, and US signal path 60 are performed in console 20. These algorithmic computations can include but are not limited to: processing multiple channels at one time, measuring propagation delays between channels, turning x, y, z data into a spatial distribution of electrode locations, including computing and applying corrections to the collection of positions, combining individual ultrasound distances with electrode locations to calculate detected endocardial surface points, and constructing a surface mesh from the surface points. The number of channels processed by console 20 can be between 1 and 500, such as between 24 and 256, such as 48, 80, or 96 channels.

A data processor 26, which can include one or more of a plurality of types of processing circuits (e.g. a microprocessor) and memory circuitry, executes computer instructions necessary to perform the processing of the pre-processed signals from the BIO signal processing module 34, localization signal processing module 44, and US TX/RX MUX 61. The data processor 26 can be configured to perform calculations, as well as perform data storage and retrieval, necessary to perform the functions of mapping subsystem 100.

In this embodiment, data processor 26 can include a bio-potential (BIO) processor 36, a localization (LOC) processor 46, and an ultrasound (US) processor 66. The bio-potential processor 36 can perform processing of recorded, measured, or sensed bio-potentials (e.g., from electrodes 12a). The LOC processor 46 can perform processing of localization signals. The US processor 66 can perform image processing of the reflected US signals, (e.g. from transducers 12b).

Bio-potential processor 36 can be configured to perform various calculations. For example, BIO processor 36 can include an enhanced common mode rejection filter, which can be bidirectional to minimize distortion and which can be seeded with a common mode signal. BIO processor 36 can also include an optimized ultrasound rejection filter and be configured for selectable bandwidth filtering. Processing steps for data in US signal path 60 can be performed by bio signal processor 34 and/or bio processor 36.

Localization processor 46 can be configured to perform various calculations. As discussed in more detail herebelow, LOC processor 46 can electronically make (calculate) corrections to an axis based on the known shape of electrode array 12, make corrections to the scaling or skew of one or more axes based on the known shape of the electrode array 12, and perform “fitting” to align measured electrode positions with known possible configurations, which can be optimized with one or more constraints (e.g. physical constraints, such as distance between two electrodes 12a on a single spline, distance between two electrodes 12a on two different splines, maximum distance between two electrodes 12a, minimum distance between two electrodes 12a, and/or minimum and/or maximum curvature of a spine, and the like).

US processor 66 can be configured to perform various calculations associated with generation of the US signal via the US transducers 12b and processing US signal reflections received by the US transducers 12b. US processor 66 can be configured to interact with the US signal path 60 to selectively transmit and receive US signals to and from the US transducers 12b. The US transducers 12b can each be put in a transmit mode and/or a receive mode under control of the US processor 66. The US processor 66 can be configured to construct a 2D and/or 3D image of the heart chamber (HC) within which the electrode array 12 is disposed, using reflected US signals received from the US transducers 12b via the US path 60.

Console 20 can also include localization driving circuitry, including a localization signal generator 28 and a localization drive current monitor circuit 29. The localization driving circuitry provides high frequency localization drive signals (e.g. 10 kHz-1 MHz, such as 10 kHz-100 kHz). Localization using drive signals at these high frequencies reduces the cellular response effect on the localization data (e.g. from blood cell deformation), and/or allows higher drive currents (e.g. to achieve a better signal-to-noise ratio). Signal generator 28 produces a high resolution digital synthesis of a drive signal, (e.g. a sine wave), with ultra-low phase noise timing. The drive current monitoring circuitry provides a high voltage, wide bandwidth current source, which is monitored to measure impedance of the patient P.

Console 20 can also include at least one data storage device 25, for storing various types of recorded, measured, sensed, and/or calculated information and data, as well as program code embodying functionality available from the console 20.

Console 20 can also include a user interface (UI) system 27 configured to output results of the localization, bio-potential, and US processing. UI system 27 can include at least one display 27a to graphically render such results in 2D, 3D, or a combination thereof. In some embodiments, the display 27a includes two simultaneous views of the 3D results with independently configurable view/camera properties, such as view directions, zoom level, pan position, and object properties, such as color, transparency, brightness, luminance, etc. UI System 27 can include one or more user input components, such as a touch screen, a keyboard, a joystick, and/or a mouse.

Console 20, or another component of mapping subsystem 100, can include one or more algorithms, such as complexity algorithm 2600 shown. Complexity algorithm 2600 can include one or more algorithms, such as one or more of: a conduction velocity algorithm, a localized rotational activity algorithm, a localized irregular activity algorithm, a focal activity algorithm, and/or another complexity algorithm. Complexity algorithm 2600 can identify, quantify, categorize, and/or otherwise assess cardiac conduction patterns or characteristics, such as to produce diagnostic information, diagnostic results 1100 herein. Complexity algorithm 2600 can produce an assessment, over time and/or space, of complexity and/or an assessment of a variation of complexity over time. In some embodiments, complexity algorithm 2600, and/or another algorithm of mapping subsystem 100, comprises a bias. In some embodiments, the algorithm comprises a bias toward false positives (e.g. a bias towards falsely identifying a non-complex region as being complex, versus not classifying a complex region as being complex). In some embodiments, the algorithm comprises a bias toward false negatives. In some embodiments, an algorithm of mapping subsystem 100 comprises a bias that is set and/or adjusted (“set” herein) by a clinician, such as to bias mapping subsystem 100 toward a particular preference of the clinician.

Complexity, as determined by the algorithms of the present inventive concepts, includes any deviation from the expected or normal behavior of what would otherwise be a simple, repetitive, and consistent pattern of electrical activity. In cardiac electrical activity, the expected or normal behavior of the heart chamber is consistent, repetitive, and coordinated activation of the tissue, called sinus rhythm, that initiates at a location (e.g. the sino-atrial node) and propagates along the chamber smoothly. Complexity includes any deviation that disrupts the consistency (e.g. time, amplitude, direction, and/or repetition rate of activation), and/or coordination/order (e.g. time and/or direction of activation). Regions of tissue may self-initiate electrical activation (automaticity), interrupting otherwise coordinated activation. Regions of tissue that may be compromised, scarred, diseased and/or possess otherwise heterogenous characteristics (e.g. fibrosis, varying fiber orientations, varying endocardial to epicardial pathways, and the like) can create complexity of cardiac activity, as described hereabove. A region that creates complexity may disrupt the expected conduction in a consistent way. For example, conduction may be redirected in a different direction and with a reduction in amplitude, but can do so in the same way for each activation. Alternatively, a region that exhibits complexity (e.g. as identified by an algorithm of mapping subsystem 100), may disrupt the expected conduction in a stochastic or probabilistic way (e.g. seemingly random variation), but in a way that possesses a recognizable statistical behavior in how it disrupts conduction. For example, modified conduction can be identified through a region in one characteristic manner for X % of the time, and in a second, different characteristic manner, for Y % of the time. In some embodiments, for Z % (where Z<100) of the time, the activation exhibits normal conduction, however the region is still identified by mapping subsystem 100 as complex due to modified conduction, in one or more forms, for some portion of the time.

The algorithms of the present inventive concepts can be configured to identify when multiple regions of complexity interact, or otherwise couple, in ways that create further complexity across the cardiac chamber, thereby compounding the degree of global complexity over the heart chamber. Because the cardiac tissue has propagative properties with a refractory (non-active) period, complexity that impacts the order and timing of activation can have lasting/persisting effects on later activations in time, and across a broad spatial area. Therefore, as the number of unique or discrete zones of automaticity or heterogeneity increases (tissue-mediated complexity), the resulting electrical activation becomes increasingly complex (e.g. a compounding of both tissue-mediated complexity and coupling-related complexity) tied together in time and space by the propagating nature of cardiac tissue, established by the variations in conduction preceding, and affecting variations in conduction to follow. As the complexity increases, the ability to identify the tissue-mediated complexity from the coupling-related complexity based on simple electrical measurements becomes more difficult. Mapping subsystem 100 can be configured to gather more information over time and across space (e.g. simultaneously), with the additional information gathered to aid in one or more algorithms decoding the complexity locally, regionally, and globally across the chamber.

Complexity algorithm 2600 can perform a complexity assessment based on calculated electrical activity data 2120b that represents multiple vertices, such as when the associated recorded electrical activity data 2120a comprises data recorded from at least three recording locations within a heart chamber (e.g. on and/or offset from the heart wall). In some embodiments, the recorded electrical activity data 2120a includes at least one location offset from the walls of the heart (e.g. at least one non-contact recording). In some embodiments, the recorded electrical activity data 2120a includes at least one location on a wall of the heart (e.g. at least one contact recording). In some embodiments, the recorded electrical activity data 2120a includes at least one location offset from the walls of the heart, and at least one location on a wall of the heart (e.g. at least one contact and one non-contact recording, a ‘hybrid’). In some embodiments, for each location on the heart wall in which a contact-based measurement is made, mapping subsystem 100 is biased to categorize that location as a vertex.

In some embodiments, algorithm 2600 comprises a second algorithm configured to calculate surface charge data and/or dipole density data for each of the multiple vertices, based on the recorded electrical activity data 2120a (e.g. recorded voltages), such as when the complexity analysis is based on surface charge data and/or dipole density data. Surface charge data and/or dipole density data can be calculated as described in U.S. Pat. No. 8,417,313, titled “METHOD AND DEVICE FOR DETERMINING AND PRESENTING SURFACE CHARGE AND DIPOLE DENSITIES ON CARDIAC WALLS”, issued Apr. 9, 2013, and U.S. Pat. No. 8,512,255, titled “DEVICE AND METHOD FOR THE GEOMETRIC DETERMINATION OF ELECTRICAL DIPOLE DENSITIES ON THE CARDIAC WALL”, issued Aug. 20, 2013, the content of each of which is incorporated herein by reference in its entirety for all purposes. In some embodiments, algorithm 2600 comprises a third algorithm that converts the surface charge data and/or the dipole density data into surface voltage data, such as when the complexity analysis is based on the surface voltage data.

In some embodiments, algorithm 2600 performs a complexity assessment over a relatively small portion of the patient's heart (e.g. a relatively small portion of a patient's heart chamber), such as a portion that represents no more than of the heart wall, such as no more than 4 cm², such as no more than 1 cm². In these embodiments, electrical activity can be recorded (e.g. by electrodes 12a) from at least three recording locations, and calculated electrical activity data 2120b determined for at least 3 vertices (as described herein). In some embodiments, the at least three recording locations comprise at least three locations on the heart wall (e.g. via a contact-based recording). In some embodiments, at least one recording location is offset from the heart wall (e.g. non-contact mapping). In some embodiments, algorithm 2600 performs the small portion complexity assessment using voltage data and/or dipole density data.

In some embodiments, algorithm 2600 performs a complexity assessment over a moderate or large portion of the patient's heart, such as a portion of the patient's heart representing at least 1 cm² of heart wall tissue (e.g. wall tissue of an atria of the heart), such as a minimum surface area of 4 cm², or 7 cm². In these embodiments, electrical activity can be recorded (e.g. by electrodes 12a) from at least 24 locations within the heart (e.g. within a single heart chamber), and calculated electrical activity data 2120b can be determined for at least 64 vertices. In some embodiments, electrical activity can be recorded from at least 24 heart wall locations (e.g. via a contact-based recording), with or without additional recordings made offset from the heart wall (e.g. in the flowing blood via a non-contact-based recording). In these embodiments, electrical activity can be recorded from at least 48 heart wall locations, or at least 64 heart locations. In some embodiments, electrical activity is recorded from both locations on the heart wall and offset from the heart wall, such as when data is recorded from at least 24, at least 48, or at least 54 contact and non-contact locations within the heart chamber. In these embodiments, calculated electrical activity data 2120b can be determined for at least 100 vertices, such as at least 500, at least 3000, and/or at least 5000 vertices.

In some embodiments, the complexity algorithm 2600 incorporates data through various depths (e.g. layers) of tissue. In thicker tissues, electrical conduction can vary through the thickness. The stretch and/or strain of the tissue can also have an impact on the conduction properties of the tissue. Measuring, recording, and/or calculating electrical data or biomechanical data through the depth of tissue can be used to improve the accuracy and/or specificity of complexity algorithm 2600. In some embodiments, surface charge density and/or dipole density is calculated through a thickness of tissue of the cardiac chamber, with the calculated data used as input to complexity algorithm 2600. In some embodiments, surface charge density and/or dipole density are determined as described in applicant's co-pending U.S. patent application Ser. No. 15/926,187, titled “DEVICE AND METHOD FOR THE GEOMETRIC DETERMINATION OF ELECTRICAL DIPOLE DENSITIES ON THE CARDIAC WALL”, filed Mar. 20, 2018, the content of which is incorporated herein by reference in its entirety for all purposes.

Complexity algorithm 2600 can assess the variation of one or more characteristics, such as electrical, mechanical, functional, and/or physiologic characteristics of the heart that vary in time, space, magnitude and/or state. Studies of cardiac behavior, function, and other characteristics, over the last several decades have yielded a substantive understanding of what is considered “normal”. Cardiac conditions such as cardiac arrhythmias exhibit variations from the norm in many ways, and these variations can be quantified, qualified, and/or otherwise assessed by complexity algorithm 2600.

In some embodiments, variations in time or temporal repetition and/or stability (e.g. measures of temporal regularity and/or irregularity) indicate the presence of a cardiac arrhythmia. Electrical characteristics (e.g. cycle length, dominant frequency, harmonic organization, fractionation or measures of waveform “energy”, Shannon entropy, waveform deflections within a time window, temporal wave recurrence, regularity) can be measured or otherwise determined by mapping subsystem 100, and included in the assessment performed by complexity algorithm 2600. Mapping subsystem 100 can determine these variables using tools such as: interval analysis; Fourier, Hilbert or other transforms; wavelet analysis; and combinations of these.

Mechanical and/or functional (“mechanical” herein) characteristics assessed by algorithm 2600 can include deflection of the heart wall over time. In some embodiments, mapping subsystem 100 determines, and algorithm 2600 assesses a combination of electrical, and/or mechanical data, such as electro-mechanical delay (e.g. which can also vary as a function of time).

In some embodiments, algorithm 2600 assesses a variation in magnitude and/or state of a characteristic determined by mapping subsystem 100. For example, electrical characteristics assessed can include an assessment of electrical activity at a cardiac surface, such as an assessment of: rms amplitude; peak-to-peak amplitude; peak-negative amplitude; and combinations of these. Mechanical characteristics assessed can include total or average deflection of the heart wall through one or more phases of the cardiac cycle. In some embodiments, a combination of electrical and mechanical data includes ratios of electrical magnitude to mechanical magnitude and/or functional efficiency.

In some embodiments, algorithm 2600 assesses a variation over space or in direction of one or more characteristics. For example, electrical characteristics assessed can include: directional bipoles formed in different directions (e.g. determined from data recorded by unipolar electrodes); conduction velocity direction; spatial wave analysis; and combinations of these. In some embodiments, a Laplacian operator can be applied to electrical activity data 2120a recorded from a multi-polar and/or omni-polar catheter to provide calculated data for algorithm 2600 to assess.

In some embodiments, algorithm 2600 assesses variations in one or more characteristics, in two or more of: time; space; magnitude; and/or state. In some embodiments, algorithm 2600 assesses two or more of these that vary simultaneously, such as a temporospatial variation. In these embodiments, algorithm 2600 can assess electrical characteristics to determine if a pattern of interest occurs (e.g. focal, rotational, irregular, directional, and/or timing patterns). Algorithm 2600 can assess temporospatial features or patterns, such as an activation sequence or conduction pattern that exhibits one or more of the following characteristics: propagation that ‘breaks out’ through a confined ‘gap’ or opening, regionally constrained pivoting re-entry, and other irregular conduction patterns (e.g. patterns that vary in time and space), rotation about a central core or obstacle, and/or focal activation spreading from a single location. Algorithm 2600 can include an assessment of changes in conduction velocity (e.g. magnitude and/or direction). Algorithm 2600 can perform any qualitative and/or quantitative analysis of one or more of these characteristics, such as to provide an assessment of complexity.

The complexity assessment provided by algorithm 2600 can comprise a binary measure of whether the complexity occurred at one or more times at each location (e.g. each vertex) assessed. The complexity assessment provided by algorithm 2600 can comprise a static level of complexity across a time period (e.g. a sum, average, median, variance, standard deviation, and/or percentile level). Static levels determined can be thresholded to calculate and/or display a subset range of the static data. The complexity assessment provided by algorithm 2600 can comprise an assessment of change in complexity over time (e.g. over one or more time periods), such as an assessment of changes in rate, frequency, degree, percentile and/or probability. Complexity algorithm 2600 can perform multiple complexity assessments in sequence, such as using a “rolling window”. The multiple complexity assessments can include an assessment of a static quantity of complexity over time.

Complexity algorithm 2600 can assess complexity (e.g. changes in complexity) and produce results (e.g. diagnostic results 1100) that are used for multiple purposes. For example, algorithm 2600 can provide an assessment of the stability and/or consistency of complexity, and/or other arrhythmogenic conditions, based on an analyzed recording duration of a few minutes or less (e.g. a duration of less than 10 minutes). The assessment can differentiate areas of consistent complexity versus transient or intermittent complexity. Regions of consistency can be correlated to specific tissue substrate characteristics. In the cardiac system, areas where the tissue substrate is anisotropic, heterogeneous, abnormal or diseased may consistently create variation and/or complexity in the electrical activity at that tissue location. However, areas of normal tissue may also see variation or other complexity (wave collisions, interference, fusion, functional block, and the like) resulting from downstream interaction of complex propagating wavefronts created by anisotropic areas of the tissue substrate. This complexity is a “functional” effect where the electrophysiological interactions of propagating waves can cause these waves to interfere or interact with one another in complex ways, often intermittently. Because cardiac tissue remains in a refractory (unable to be re-activated) state for a period of time following each activation, the functional effect occurs not only at the moment when a wave of activation passes, but for an extended period after it has passed. The net result is that complexity of cardiac tissue activation, as identified by complexity algorithm 2600, can also occur in areas where the tissue itself is not abnormal or diseased, but is rather due to the prior complex interactions that occurred at other tissue locations. Fixed, substrate-mediated complexity (or mechanisms) will probabilistically re-occur at the same location. Functional complexity may vary in location and frequency of occurrence at a given location. Complexity algorithm 2600 can be configured to assess the consistency, stability, repeatability, and/or pattern of complexity to differentiate between fixed, substrate-mediated complexity vs. functional complexity.

Complexity algorithm 2600 can be used to determine electrical changes resulting from a delivered therapy (e.g. an RF or other cardiac ablation, such as a therapy provided by treatment subsystem 800, as described herebelow). Comparison of complexity and/or consistency of complexity (“complexity” herein) before and after a therapeutic activity or interval can be used to indicate the electrophysiological impact of the delivered therapy. Algorithm 2600 can provide a comparison in the form of a difference plot. Therapeutic events may be as short as a few seconds (at a single or small number of locations) or up to many minutes (for more extensive maneuvers such as ablative lines, loops, cores, boxes, and the like). The longer the therapeutic activity or interval, the more change may exist in the comparison. In some embodiments, mapping subsystem 100 provides a real time (e.g. during therapy) feedback-loop of cause (therapy) and effect (complexity assessment, such as a change in complexity prior to and after therapy). Mapping subsystem 100 can be configured to provide a complexity assessment (e.g. record electrical activity data 2120a and calculate complexity via algorithm 2600) in a relatively short period of time (e.g. less than 10 minutes, or less than 5 minutes), such that the clinician is more likely to reduce therapeutic interval times to assess complexity after each interval. In these embodiments, unnecessary ablations can be avoided and/or overall procedure time can be reduced.

Complexity algorithm 2600 can be configured to produce complexity data (e.g. the output of a complexity assessment) in real time, such that the complexity data (e.g. diagnostic results 1100) can be shown dynamically, also in real time. For example, mapping subsystem 100 can record and process electrical activity data 2120a, and algorithm 2600 can analyze the recorded activity, such as using a rolling window, such as a time window with a duration of between 5 seconds and 60 seconds. Algorithm 2600 provides multiple complexity assessments by continuously analyzing electrical activity data 2120a over the total duration assessed, with newer data added and oldest data excluded as the electrical activity data 2120a recording continues. Complexity assessments (e.g. multiple complexity assessments provided in a video format) can be provided in real time (e.g. with a short processing delay), such as during a treatment (e.g. ablation) to dynamically determine when the treatment has achieved a desired result (e.g. sufficient energy has been delivered to cause the desired effect, such as electrical block), and/or how to modify the therapy to achieve a therapeutic goal or otherwise improve efficiency. Alternatively or additionally, the provided complexity assessments can be visualized (e.g. in a playback mode) one or more times after the associated recording of electrical activity data 2120a has ceased, such as to perform additional therapy and/or modify the therapy.

Complexity algorithm 2600 can provide complexity assessments based on electrical activity data 2120 (and/or additional patient data 2150 as described herebelow) recorded during two separate clinical procedures (e.g. a first clinical procedure and a subsequent, second clinical procedure). Algorithm 2600 can provide one or more complexity assessments for each clinical procedure, such as to allow a comparison to be made between assessments from two different procedures (e.g. an assessment made by algorithm 2600). The second clinical procedure can be separated from the first clinical procedure by days, weeks, months, or years. A comparative assessment made by algorithm 2600 can assess the therapeutic effects of the first procedure and the recovery (e.g. healing) of the cardiac tissue or the adaptation of the cardiac tissue in the interim between procedures. Cardiac tissue may adapt in response to the altered electrical characteristics (e.g. altered patterns, rhythms, and the like, such as from electrical remodeling), and/or the altered mechanical characteristics (e.g. function) of the tissue, each as caused by the preceding therapeutic procedure. Techniques used in the second clinical procedure can be based on these above assessments provided by algorithm 2600 (e.g. in the form of diagnostic results 1100), such as the tissue response (e.g. the electrical and mechanical response described hereabove) to the therapy provided in the first procedure.

While algorithm 2600 has been described hereabove as analyzing electrical activity data 2120, in some embodiments, algorithm 2600 further includes in its assessment, an analysis of “additional patient data” recorded by mapping subsystem 100 (e.g. the complexity assessment is based on additional patient data 2150 recorded by mapping subsystem 100 as well as electrical activity data 2120 and anatomical data 2110 described hereabove). For example, mapping subsystem 100 can comprise one or more functional elements configured as sensors, such as functional element 99 of mapping catheter 10, functional element 899 of treatment catheter 800 described herebelow, and/or functional element 199 of mapping subsystem 100. Functional element 99 of mapping catheter 10 can comprise one or more sensors positioned on an expandable spline of electrode array 12 (as shown), and/or on shaft 16. Functional element 199 of mapping subsystem 100 can comprise a sensor positioned proximate the patient (e.g. on the skin of the patient or relatively near the patient) and/or a sensor positioned within the patient (e.g. temporarily or chronically positioned under the patient's skin). In some embodiments, one or more electrodes 12a and/or ultrasound transducers 12b are configured to record the additional patient data 2150.

In some embodiments, sensor-based functional elements 99, 199, and/or 899 comprises a sensor selected from the group consisting of: an electrode or other sensor for recording electrical activity; a force sensor; a pressure sensor; a magnetic sensor; a motion sensor; a velocity sensor; an accelerometer; a strain gauge; a physiologic sensor; a glucose sensor; a pH sensor; a blood sensor; a blood gas sensor; a blood pressure sensor; a flow sensor; an optical sensor; a spectrometer; an interferometer; a measuring sensor, such as to measure size, distance, and/or thickness; a tissue assessment sensor; and combinations of one, two, or more of these.

Additional patient data recorded by mapping subsystem 100 (e.g. via mapping catheter 10, functional element 199, functional element 899, and/or other sensor of system 10), can include patient mechanical information; patient physiologic information, and/or patient functional information. Additional data recorded by mapping subsystem 100 can include data related to a patient parameter selected from the group consisting of: heart wall motion; heart wall velocity; heart tissue strain; magnitude and/or direction of heart blood flow; vorticity of blood; heart valve mechanics; blood pressure; tissue properties, such as density, tissue characteristics and/or biomarkers for tissue characteristics, such as metabolic activity or pharmaceutical uptake; tissue composition (e.g. collagen, myocardium, fat, connective tissue); and combinations of one, two, or more of these.

As described hereabove, one or more complexity assessments performed by algorithm 2600 can be based on this additional patient data, such as when both electrical activity data 2120 and additional patient data 2150 is included in the analysis performed. In some embodiments, the complexity assessment performed by algorithm 2600 comprises an assessment of one or more of: electrical-mechanical delay of tissue; magnitude ratio of an electrical to a mechanical characteristic; and combinations of these.

Additional patient data 2150 can also comprise prior data (e.g. data collected during a prior procedure) from the same patient or prior data from a set of historical patients other than the patient being diagnosed or treated. The data can be used to form a computational model into which the existing patient's data is fitted, classified, ranked, prioritized, optimized, and/or otherwise assessed as described above.

Diagnostic results 1100 can comprise measured data and/or data resulting from an analysis of measured data (e.g. an analysis of electrical activity data 2120a and/or anatomical data 2110). Diagnostic results 1100 can be provided (e.g. provided to a clinician of the patient), in one or more forms, such as when displayed on display 27a, provided audibly (e.g. by a speaker of mapping subsystem 100), and/or provided in a printed report (e.g. by a printer of mapping subsystem 100). Diagnostic results 1100 can be used by a clinician to customize a therapy for the patient, such as to determine at which locations to ablate tissue in a cardiac ablation procedure, such as is described in applicant's co-pending U.S. patent application Ser. No. 14/422,941, titled “CATHETER, SYSTEM AND METHODS OF MEDICAL USES OF SAME, INCLUDING DIAGNOSTIC AND TREATMENT USES FOR THE HEART”, filed Feb. 20, 2015, the content of which is incorporated herein by reference in its entirety for all purposes.

In some embodiments, diagnostic results 1100 are based on a complexity assessment performed by complexity algorithm 2600 for a single heart wall location or multiple heart wall locations. The single and/or multiple location diagnostic results 1100 can be presented to a user (e.g. the patient's clinician) in reference to an image of the patient's anatomy (e.g. via display 27a). Diagnostic results 1100 can comprise an assessment of complexity over time, such as an assessment of complexity over a pre-determined time duration.

As described hereabove, mapping subsystem 100 can be configured to perform a medical procedure (e.g. a diagnostic, prognostic, and/or therapeutic procedure) related to an arrhythmia or other cardiac condition of the patient. Mapping subsystem 100 can be configured to perform a medical procedure on a patient with a cardiac condition selected from the group consisting of: atrial fibrillation; atrial flutter; atrial tachycardia; atrial bradycardia, ventricular tachycardia; ventricular bradycardia; ectopy; congestive heart failure; angina; arterial stenosis; and combinations of one, two, or more of these. In some embodiments, mapping subsystem 100 performs a medical procedure on a patient that exhibits heterogeneous activation, conduction, depolarization, and/or repolarization that varies in time, space, magnitude, and/or state (e.g. combinations, such as velocity). Electrical activity of the patient's heart may contain patterns that can be detected or mapped by system 10, such as patterns selected from the group consisting of: focal; re-entrant; rotational; pivoting; irregular (e.g. in direction and/or velocity); functional block; permanent block; and combinations thereof.

Mapping subsystem 100 can include devices or agents (e.g. pharmaceutical agents), treatment subsystem 800, for treating a patient (e.g. treating one or more cardiac conditions of the patient). In the embodiment shown in FIG. 2, treatment subsystem 800 includes a treatment catheter 850, including shaft 860, which can be configured to be advanced through the patient's vasculature into one or more chambers of the patient heart, using standard interventional techniques. In some embodiments, the distal portion of shaft 860 is advanced into the patient's left atrium via a transseptal sheath, not shown but such as a standard device used in left atrial ablation procedures. Treatment catheter 850 comprises treatment element 870 on the distal end (as shown) or at least the distal portion of shaft 860. Treatment element 870 can comprise one or more treatment elements, such as one or more energy delivery elements configured to deliver energy to ablate cardiac tissue (e.g. ablation energy delivered to the heart wall). Treatment element 870 can include an array (e.g. a linear or other array) of treatment elements. Treatment element 870 can comprise one or more electrodes configured to deliver radiofrequency (RF) or other electromagnetic energy to tissue. In some embodiments, treatment element 870 comprises one or more energy delivery elements configured to deliver energy in a form selected from the group consisting of: electromagnetic energy such as RF energy and/or microwave energy; thermal energy such as heat energy and/or cryogenic energy; light energy such as laser light energy; sound energy such as ultrasound energy; chemical energy; mechanical energy; and combinations of these. In some embodiments, treatment element 870 comprises one or more agent delivery elements (e.g. one or more needles, iontophoretic elements, and/or fluid jets) configured to deliver an agent (e.g. a pharmaceutical agent) into cardiac tissue or other tissue of the patient.

Treatment subsystem 800 can further include an energy delivery unit, EDU 810 which provides energy to the one or more treatment elements 870. EDU 810 can provide one or more form of energy selected from the group consisting of: electromagnetic energy such as RF energy and/or microwave energy; thermal energy such as heat energy and/or cryogenic energy; light energy such as laser light energy; sound energy such as ultrasound energy; chemical energy; mechanical energy; and combinations of these. Alternatively or additionally, EDU 810 can provide an agent to one or more treatment elements 870, such as when treatment elements 870 comprise an agent delivery element as described hereabove.

In some embodiments, treatment subsystem 800, treatment catheter 850, and/or EDU 810 are of similar construction and arrangement to the similar components described in applicant's co-pending U.S. patent application Ser. No. 14/422,941, titled “CATHETER, SYSTEM AND METHODS OF MEDICAL USES OF SAME, INCLUDING DIAGNOSTIC AND TREATMENT USES FOR THE HEART”, filed Feb. 20, 2015, the content of which is incorporated herein by reference in its entirety.

In some embodiments, treatment subsystem 800 is used to treat the patient based on the diagnostic results 1100 (e.g. results which are based on complexity assessment provided by algorithm 2600). For example, ablation energy can be delivered to the heart wall at one or more locations (e.g. one or more vertexes described hereabove), where the complexity assessment determines if a complexity level for a location exceeds (e.g. is above) a threshold, and therapy is delivered to all locations where the threshold is exceeded. In some embodiments, one vertex is selected for ablation, in a region of multiple vertices, where mapping subsystem 100 (e.g. via algorithm 2600) determines a maximum complexity level to exist (e.g. a “local maximum” is ablated), and where the maximum complexity level can be an absolute maximum or a relative maximum.

In some embodiments, therapy provided by mapping subsystem 100 (e.g. ablation energy delivered to one or more vertices) is delivered in a closed-loop fashion, such as in a manual (clinician driven), automated (e.g. mapping subsystem 100 driven), and/or semi-automated (e.g. combined clinician and mapping subsystem 100 driven) mode. Closed-loop operation can include: manipulation of treatment element 870 to a location to be treated (e.g. via clinician manipulated and/or mapping subsystem 100 robotically manipulated treatment device 850); and/or setting of energy level to be delivered.

Referring now to FIG. 3, a flow chart of a method of processing cardiac information is illustrated. consistent with the present inventive concepts. Method 2000 of FIG. 3 is described using system 1000 and its components as described hereabove in reference to FIGS. 1 and/or 2.

In STEP 2010 data is recorded from the patient, such as data recorded using mapping catheter 10 and/or other components of system 1000, as described hereabove.

In some embodiments, the recorded data comprises a plurality of biopotential signals, for example biopotential signals recorded from one or more electrodes (e.g. mapping data 110). The recording electrodes can comprise one or more electrodes positioned inside the patient, such as one or more electrodes 12a of mapping catheter 10. In some embodiments, the recording electrodes can be positioned inside a heart chamber (e.g. endocardial) and/or outside the heart chamber (e.g. epicardial). Additionally or alternatively the recording electrodes can comprise one or more electrodes positioned on the skin of the patient, such as one or more patch electrodes 56.

In some embodiments, the recorded data comprises imaging data 210, such as CT or MRI data.

In STEP 2020 one or more algorithms of system 1000 (e.g. data processing algorithm 551, option algorithm 552, and/or learning algorithm 553) perform an analysis of the data recorded in STEP 2010.

In some embodiments, data processing algorithm 551 can comprise a self-improving algorithm (e.g. such as an improvement achieved via learned information generated by learning algorithm 553). Data processing algorithm 551 can be configured to analyze a “present” activation (e.g. an activation being recorded in near real-time), and it can predict the path of the activation across an endocardial surface. As used herein, a pattern of activation “across an endocardial surface” can include activation throughout a surface and/or volume of cardiac tissue, such as tissue between the endocardial and epicardial surfaces (e.g. the cardiac wall). A pattern of activation can include an activation at a point, activation within a volume, a line of activation traversing a straight or curved path, and/or a plane of activation traversing a flat or curved plane. As additional data is recorded, algorithm 551 can be configured to adjust the predictions based on the additional data. Learning algorithm 553 can be configured to analyze differences between the predicted outcomes and the recorded outcomes. This analysis can be stored as learned data 557 and can be used to improve and refine the accuracy of predictive processing performed by data processing algorithm 551. In some embodiments, this refinement can be achieved using one or more of: artificial intelligence, machine learning, and/or deep learning configurations.

In some embodiments, data processing algorithm 551 can perform a statistical analysis method to identify one or more preferential conduction pathways of activation across the endocardial surface. In some embodiments, any identified preferential conduction pathways can be incorporated into the predictive algorithm described hereabove.

In some embodiments, option algorithm 552 comprises a predictive algorithm, such as an algorithm configured to predict the effect of one or more treatments (e.g. predict the effect of one or more created lesions) on the pattern of activation across a heart surface (e.g. an endocardial heart chamber surface). For example, option algorithm 552 can be configured to predict the effect of an RF ablation on the recorded pattern of activation. Option algorithm 552 can comprise an iterative search algorithm (e.g. a recursive algorithm), such as an iterative algorithm configured to simulate, predict the effects of, and assess the outcomes of one or more treatment strategies. In some embodiments, option algorithm 552 comprises an iterative search algorithm configured to predict treatment parameters (e.g. RF or other ablation parameters), such as desired (e.g. optimized) ablation locations and/or a number (e.g. a minimum number) of treatments (e.g. ablations), such as to efficiently and/or effectively treat an abnormal rhythm or other undesired cardiac condition (e.g. to convert an arrhythmia to normal sinus rhythm in reduced steps and/or with improved efficacy). In some embodiments, option algorithm 552 is configured to analyze more than 10,000 outcomes, such as more than 100,000 outcomes, in order to determine an optimal treatment strategy. As a treatment strategy is performed and additional data is recorded, algorithm 552 can be configured to adjust the predicted parameters of the treatment strategy based on the additional data. Learning algorithm 553 can be configured to analyze the differences between the predicted outcomes of the treatment strategy and the recorded outcomes. This analysis can be stored as learned data 557, and it can be used to improve and refine the accuracy of predictive processing of option algorithm 552. In some embodiments, data processing algorithm 551, option algorithm 552, and/or learning algorithm 553 are configured to analyze raw mapping data, such as biopotential data recorded from one or more electrodes 12a, and/or to analyze processed data, such as mapping data 110 comprising data calculated using an inverse solution to determine dipole density and or surface charge density and/or other mapping data 110 comprising data calculated using one or more post-processing algorithms.

One or more algorithms of system 1000 (such as data processing algorithm 551 and/or option algorithm 552) can be configured to perform predictive processing based on a functional model of the cardiac anatomy, functional model 559. The functional model 559 can comprise one or more functional rules configured to parameterize the predictive processing performed by one or more algorithms. In some embodiments, functional model 559 can comprise a refractory time parameter (e.g. a time duration between a localized cardiac activation and when the activated area can be activated a subsequent time). In some embodiments, the refractory time parameter is dependent on the cycle length interval preceding the current cycle (e.g. the cycle currently being modeled by functional model 559). In some embodiments, the refractory time parameter is frequency dependent. In some embodiments, the refractory time parameter is modified by simulating the effects of one or more medications. In some embodiments, the refractory time parameter varies based on the region of cardiac tissue being modeled, such as a variation based on the thickness of the tissue, the density of the tissue, the heterogeneity of the tissue, the percentage of fibrosis of the tissue, the number of trabeculated muscles in posterior versus anterior locations, and/or for the septum of the atrium. In some embodiments, the refractory time parameter is adjusted based on the volume and/or the pressure within the heart chamber, for example such that the refractory time parameter is longer during the ventricular stroke, and/or shorter during systole.

In some embodiments, functional model 559 can comprise one or more parameters selected from the group consisting of: the size and/or location of the pulmonary veins; the size, location, and/or other parameters of one or more cardiac valves; the size and/or shape of one or more cardiac chambers; the thickness of the walls of one or more cardiac chambers; the size and/or location of the atrial appendage; and combinations of one or more of these. In some embodiments, functional model 559 can comprise a triangular and/or a quadratic mesh.

In some embodiments, data processing algorithm 551 is configured to assess the amplitude and/or morphology of a recorded biopotential signal in an area (e.g. a volume) of the cardiac tissue, such as to predict one or more tissue characteristics within that area. For example, an area with a low recorded signal amplitude can be predicted to comprise an area of slow conduction and/or scar tissue. Additionally or alternatively, an area with a high recorded signal amplitude can be predicted to comprise healthy tissue. In some embodiments, a change in recorded signal amplitude for a location (e.g. a change observed between two recordings made prior to and after a treatment, respectively) can be analyzed to predict the effectiveness of the treatment (e.g. the extent and/or the transmurality of an ablation treatment in said location). In some embodiments, option algorithm 552 is configured to preferentially assess treatment options including treating certain areas, such as areas of low recorded signal amplitude (for example when iteratively searching for an optimal treatment strategy, as described hereabove). Areas of low recorded signal amplitude may comprise tissue which is less thick, and therefore more easily treated, such as with ablation (e.g. RF ablation). Alternatively or additionally, option algorithm 552 can be configured to preferentially avoid certain areas, such as the posterior wall of the left atrium and/or other areas which may be difficult to treat.

In some embodiments, data processing algorithm 551 is configured to assess the morphology of data recorded by system 1000. In some embodiments, evaluation data 556 can correlate one or more morphologies with an electrical property of the tissue area assessed. For example, a negative signal can be correlated with a centrifugal activation, a positive signal can be correlated with an approaching wavefront, a positive signal followed by a negative signal can be correlated with a passing wavefront, and/or a negative signal followed by a positive signal can be correlated with a “mirror image activation wavefront from opposite site”. In some embodiments, evaluation data 556 can correlate opposing vectors with the collision of activation wavefronts. In some embodiments, evaluation data 556 can correlate opposite vectors with a negative and a positive component with the incomplete block of a line. In some embodiments, evaluation data 556 can correlate positive vectors (e.g. R-wave) along a line with no negative component with a complete block of a line. In some embodiments, evaluation data 556 can correlate reverse polarity of signal around a point in opposite directions with a focal activation at that point. In some embodiments, evaluation data 556 can correlate the loss of the negative component (e.g. Q-S-wave) of an electrical signal (e.g. a dipole density signal or a voltage signal) with a transmural ablation.

In some embodiments, a “mirror image S-R signal” (i.e. opposite of “R-S”) can correlate to an error in data processing algorithm 551. If the algorithm is not properly implemented, geometrically symmetric “mathematical modes” of spatial “ringing” can be generated (e.g. a spatial manifestation of the “Gibbs phenomenon”). For example, there can be a large “ringing-mode” situated at 180 degrees from the location of the “real” electrical event, sometimes referred to as a “mirror potential”. Data processing algorithm 551 can be configured to identify errors such as described hereabove, and to self-correct, such as by altering the parameters of the inverse solution. In some embodiments, a “R-S” signal can correlate to activations occurring independently and simultaneously, generally on opposite sides of a cardiac chamber. If the magnitude of a signal is large enough to “influence” a measurement on the opposite side of a chamber, then it will be recorded as such. In some embodiments, it is possible for there to be no local event, and Q-S or “R-S” morphologies are only representative of a far-field signal. It can be that there is a simultaneous local event, which would then “blend” with the far-field event.

In some embodiments, mapping console 20 is configured to produce mapping data 110 comprising data calculated using an inverse solution method, comprising one or more constraint parameters. For example, mapping data 110 can comprise dipole density and/or surface charge density data, calculated by algorithm 120 of mapping console 20 using an inverse method such as a method using Poisson's equation, a fundamental theorem in electrostatic field theory that relates a distribution of charge to the voltage it generates both surrounding and within the distribution of charge. In some embodiments, data 110 is calculated using the systems and methods described in U.S. Pat. No. 8,417,313, titled “METHOD AND DEVICE FOR DETERMINING AND PRESENTING SURFACE CHARGE AND DIPOLE DENSITIES ON CARDIAC WALLS”, issued Apr. 9, 2013, and U.S. Pat. No. 8,512,255, titled “DEVICE AND METHOD FOR THE GEOMETRIC DETERMINATION OF ELECTRICAL DIPOLE DENSITIES ON THE CARDIAC WALL”, issued Aug. 20, 2013, the contents of each of which are incorporated herein by reference in their entirety for all purposes. An inverse solution requires boundary conditions and regularization parameters to be set by system 1000 in order to perform the calculation of the solution and produce the desired data. Adjusting these boundary conditions and regularization parameters, the parameters of the inverse solution can improve the accuracy of the produced data. For example, the inverse solution can constrain the dipole density and/or the surface charge calculated to be only within the myocardium of the heart chambers. In some embodiments, one or more algorithms of system 1000, such as algorithm 120, data processing algorithm 551, and/or learning algorithm 553 are configured to optimize the parameters of the inverse solution. For example, data processing algorithm 551 can comprise an iterative algorithm configured to model an activation pattern based on mapping data 110, modify one or more of the inverse solution parameters, recalculate mapping data 110 using the new parameters, and remodel the activation pattern. This iterative method can be used to determine the optimal inverse solution parameters to provide a stable model of activation (e.g. the most stable model of activation). In some embodiments, mapping data 110 comprises both dipole density data calculated from non-contact recordings and voltage measurements recorded from contact recordings (e.g. from electrodes in contact with the cardiac wall). Algorithm 120 and/or data processing algorithm 551 can be configured to compare the calculated dipole density data to the voltage measurements, and to modify the parameters of the inverse solution to correct any discrepancies identified between the calculated data and the measured data.

In some embodiments, patient data 521 comprises information relating to a patient's medication (e.g. medication the patient takes regularly and/or medication given to the patient during a procedure). Learning algorithm 553 can be configured to integrate this medication information into learned data 557. Option algorithm 552 can be configured to analyze this information to predict the efficacy of medication, such as the efficacy of a medication on a current patient, based upon learned data 557 gathered from a prior patient population (e.g. one or more, such as tens of thousands of prior patients). In some embodiments, learned data 557 can comprise information related to the effectiveness of one or more ablation or other tissue treatment procedures (such as one or more ablation procedures performed on one or more prior patients, such as tens of thousands of prior patients). Option algorithm 552 can be configured to analyze this information and predict the efficacy of a treatment procedure, such as an RF ablation. In some embodiments, this prediction data can be used to screen patients, such as to prevent unsuccessful RF ablation or other treatment procedures. In some embodiments, learning algorithm 553 can analyze training data 520, including patient data 521, procedure data 522, and outcome data 523. Patient data 521 can comprise age, atrial diameter, and/or size of electrograms.

In some embodiments, data processing algorithm 551 is configured to predict atrial activation wavefronts, such as by performing a frequency analysis on mapping data 110 (e.g. such as a Fourier transformation). This analysis can include determining a dominant frequency and/or cycle length. In some embodiments, the frequency analysis can include the determination of: a dominant frequency; a frequency ratio; entropy; organization index; energy of the signal; power of the signal; and combinations of one or more of these. Data processing algorithm 551 can be configured to predict the length of a conduction circuit based off of an analysis of a determined cycle length and/or measured conduction velocity.

In STEP 2030 the patient is treated, such as using therapeutic device 350 and/or other components of system 1000 as described hereabove in reference to FIGS. 1 and/or 2. STEP 2030 can be performed prior to, during, and/or after STEP 2010. For example, STEP 2020 can be performed after at least a portion of the treatment of STEP 2030 is performed, such as to assess the treatment and/or adjust future treatments. For example, data recorded during a prior procedure (e.g. a previous mapping and/or treatment procedure) can be analyzed during STEPS 2010 and/2020.

In STEP 2040, system 1000 is configured to display data to the user, such as evaluation data 556 displayed on display 532, as described hereabove in reference to FIGS. 1 and/or 2.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

What is claimed is:

1. A cardiac information processing system, comprising:
multiple subsystems for performing a procedure and producing procedure data, the multiple subsystems comprising:
a mapping subsystem comprising at least one mapping catheter;
an imaging subsystem comprising at least one imaging device; and
a treatment subsystem comprising at least one treatment device; and
a processing module for receiving the procedure data, and comprising at least one processor and at least one algorithm, wherein the at least one algorithm is configured to:
perform an assessment of the procedure data and produce evaluation data based on the assessment.
